(19) United States
(12) Patent Application Publication
(10) Pub. No.: US 2022/0378774 A1
(43) Pub. Date: Dec. 1, 2022

(54) METHOD OF PROMOTING CELLULAR HYDRATION BY ENHANCING INTRACELLULAR PERMEATION

(71) Applicant: Eastpond Laboratories Limited, Douglas (IM)

(72) Inventor: Lajos SZENTE, Budapest (HU)

(73) Assignee: Eastpond Laboratories Limited, Douglas (IM)

(21) Appl. No.: 17/884,516

(22) Filed: Aug. 9, 2022

Related U.S. Application Data
(63) Continuation of application No. 16/841,646, filed on Apr. 6, 2020, now abandoned.
Application No. 16/841,646 is a continuation-in-part of application No. 14/932,929, filed on Nov. 4, 2015, now Pat. No. 10,610,524.
Application No. 14/932,929 is a continuation of application No. 12/983,234, filed on Dec. 31, 2010, now abandoned.

(51) Int. Cl. A61K 31/455; A61K 31/198; A61K 9/00; A23L 2/58; A23L 2/56; A23L 2/38; A23L 29/212; A23L 33/175; A23L 33/15; C08L 5/16; A23L 2/52; A23L 2/66; A61K 47/61

(52) U.S. Cl. CPC A61K 31/455; A61K 31/198; A61K 9/0095; A23L 2/58; A23L 2/56; A23L 2/38; A23L 29/212; A23L 33/175; A23L 33/15; C08L 5/16; A23L 2/52; A23L 2/66; A61K 47/61; A23V 2002/00

(57) ABSTRACT

A method of promoting increased cellular hydration in a multicellular organism that is capable of intracellular water permeation includes the step of causing the multicellular organism to ingest an aqueous solution that contains an amount of a carbohydrate clathrate component. There is also a step of enhancing the intracellular permeation. The multicellular organism contains aquaporins, and the causing step involves interaction of the composition with the aquaporins. The cellular hydration promoted and caused by the method is corroborated by a test that uses human-aquaporin-expressed frog oocytes. The test uses single cell Xenopus laevis frog oocytes having expressed human aquaporin AGP1 water channels. There is also a beverage composition that increases lifespan in the multicellular organism, and a beverage composition that promotes cellular hydration when ingested by a multicellular organism.

### CROSS-REFERENCE TO RELATED APPLICATIONS

This application is a continuation of U.S. patent application Ser. No. 16/841,646, filed Apr. 6, 2020, which application is a continuation-in-part of U.S. patent application Ser. No. 14/932,929, filed Nov. 4, 2015, now U.S. Pat. No. 10,610,524, which application is a continuation of U.S. patent application Ser. No. 12/983,234, filed Dec. 31, 2010, the disclosures of which are incorporated herein by reference.

### FIELD OF THE INVENTION

The present invention generally relates to regulation of biological cell activity, particularly cell activity dependent on hydration state. A biologically active component is constructed to increase an activity of a biological cell system by increasing the hydration of one or more components of that cell system. That biologically active component may include a primary carbohydrate clathrate subcomponent that increases the H-bonded structure of water. More particularly, the present invention relates to a beverage composition comprising the biologically active component for increasing the cell hydration and consequently modifying physiological activity of multicellular organisms, including mammals. Furthermore, the present invention relates to a mechanism of action for increasing cellular hydration in multicellular organisms, including mammals.

### BACKGROUND OF THE INVENTION

Water molecules interact principally through hydrogen (H)-bonding and through alignment of dipole moments. For example, bonds between neighboring water molecules are reinforced, or stabilized, by alignment of bond axes with next-adjacent water molecules. In liquid state water, such alignments propagate into the surrounding aqueous medium and establish sub-micrometer scale molecular structure.

Examples of products and methods of using cyclodextrins as clathrates to form inclusions with bioactive guest molecules to improve solubility and/or bioavailability of pharmaceutical compounds are described in: U.S. Pat. Nos. 7,115,586 and 7,202,233, and U.S. Patent Application Publication Nos. 2004/0137625, and 2009/0227690, the complete disclosures of which are hereby incorporated by reference for all purposes.

Examples of products and methods of using products containing clathrates that bind hydrophobic biomolecules are described in U.S. Pat. Nos. 6,890,549, 7,105,195, 7,166,575, 7,423,027, and 7,547,459; U.S. Patent Application Publication Nos. 2004/0161526, 2007/0116837, 2008/0299166, and 2009/0023682; Japanese Patent Application JP 60-094912; Suzuki and Sato, “Nutritional significance of cyclodextrins: indigestibility and hypolipemic effect of α-cyclodextrin” J. Nutr. Sci. Vitaminol. (Tokyo 1985; 31:209-223); and Szejtli et al., Staerke Starch, 27(11), 1975, pp. 368-376, the complete disclosures of which are hereby incorporated by reference for all purposes.

U.S. Patent Application Publication No. 2009/0110746 describes chemical agents which have the property of increasing aqueous diffusivity of dissolved molecular oxygen (O₂) in the human body, wherein cyclodextrins may be included as secondary “carrier” components to improve the solubility of primary pro-oxygenating agents, and wherein cyclodextrins are not contemplated as agents to directly alter aqueous diffusivity, tissue oxygenation, water structure, or cellular hydration.

Also, Park et al. (2013) describes effect of type of water on the life span extension of C. elegans. Similarly, Gelino et al. (2016) describes longevity in C. elegans with respect to functions for autophagy in the intestine of dietary-restricted C. elegans (also known as Caenorhabditis elegans) and water absorption.

The present invention overcomes the drawback of conventional compositions, systems and methods.

### BRIEF DESCRIPTION OF THE DRAWINGS

FIG. 1 shows a chemical bond model of β-cyclodextrin, a cyclic oligosaccharide having seven α[1-4] linked glucose units.

FIG. 2 shows a structural model of cyclodextrins having an overall toroid topology.

FIG. 3 shows a cyclodextrin structural model including the disposition of glucosyl hydroxyl groups along the toroid rims.

FIG. 4 depicts a calculated molecular dynamic distribution of water molecules surrounding a β-cyclodextrin molecule at 1 picosecond after initial contact.

FIG. 5 depicts the calculated molecular dynamic distribution of water molecules of FIG. 4 at 1000 picoseconds after initial contact, including a more organized open water structure.

FIG. 6 shows threshold images of the water molecule distributions shown in FIGS. 4 and 5.

FIGS. 7-10 show a comparison of NR spectra derivatives, including particular wavelength regions, for water samples with and without dissolved cyclodextrins.

FIG. 11 shows a comparison of seed germination kinetics in water variably including a cyclodextrin, an amino acid, and a cyclodextrin/amino acid inclusion complex.

FIG. 12 shows a comparison of seed germination kinetics in water variably including a cyclodextrin, a vitamin, and a cyclodextrin/vitamin inclusion complex.

FIG. 13 shows a comparison of seed germination rate in water variably including active components of hydration according to the present disclosure.

FIG. 14 shows a comparison of nematode longevity in media variably including cyclodextrins as an active component of hydration according to the present disclosure.

FIG. 15 shows a comparison of nematode longevity in media variably including derivatized cyclodextrins as an active component of hydration according to the present disclosure.

FIG. 16 shows a comparison of nematode longevity in media variably including cyclodextrin inclusion complexes as an active component of hydration according to the present disclosure.

FIG. 17 shows nematode mortality frequency in media with and without a cyclodextrin inclusion complex included as an active component of hydration according to the present disclosure.

FIG. 18 shows population survival curves for nematodes living in media with and without a cyclodextrin inclusion complex as an active component of hydration according to the present disclosure.

FIG. 19 shows lipid bilayer representing arrangement of phospholipids, membrane proteins, cholesterol, functional proteins etc. within the lipid bilayer.

FIG. 20 shows water paracellular transport.

FIG. 21 shows the effect of 0.1% alpha-cyclodextrin containing water versus a control (plain water with no additive).

FIG. 22a shows the effect of 0.1% Alpha-CD, 0.1% alpha-CD-nicotinic acid complex, and 0.1% alpha-CD-arginine complex on the lifespan of C. elegans.

FIG. 22b shows the effect of 0.5% of alpha-cyclodextrin and 0.05% alpha-cyclodextrin containing water versus control (plain water with no additives) on the lifespan of C. elegans.

FIG. 22c shows the effect of 0.05% of alpha-cyclodextrin-nicotinic acid complex and 0.05% of alpha-cyclodextrin-L-arginine complex, versus control (no additives in water) on the lifespan of C. elegans.

FIG. 23 shows human-aquaporin-expressed frog oocyte osmotic water permeability according to the present disclosure. A calibration oocyte on the right of the photo

FIG. 24 shows human-aquaporin-expressed frog oocyte osmotic water permeability according to the present disclosure. The control un-swollen small oocyte is on the right of each photo

FIGS. 25A and 25B show the osmotic water permeability (Pf values) of human-aquaporin-expressed frog oocytes in two-time scales according to the present disclosure; wherein C1: control (purified water), C2-C4: ACD 0.05%, 0.1%, 0.5%; C5-C7: ACD-nicotinic acid complex, 0.05%, 0.1%, 0.5%, C8-C10: ACD-L-arginine complex, 0.05%, 0.1%, 0.5%.

### SUMMARY OF INVENTION

The present invention provides a method of promoting increased cellular hydration in a multicellular organism that is capable of intracellular water permeation. The method includes the step of causing the multicellular organism to ingest an aqueous solution that contains an amount of a carbohydrate clathrate component; and enhancing the intracellular permeation. The multicellular organism contains aquaporins, and the causing step involves interaction of the composition with the aquaporins. The cellular hydration promoted and caused by the method is corroborated by a test that uses human-aquaporin-expressed frog oocytes. The test uses single cell Xenopus laevis human-aquaporin-expressed frog oocytes having expressed human aquaporin AGP1 water channels.

Continuing with the summary, the multicellular organism has lipid bilayer constituents, and the method also involved forming non-covalent inclusion complexes between the clathrate component and the lipid bilayer constituents. The multicellular organism also has phospholipids, which may be linear, and may be glycosphingolipids, sphingomyelin, phosphatidylcholine, phosphatidyl ethanolamine. The multicellular organism also includes membrane lipids and proteins, and the causing step results in reversible and temporary disintegration of membrane lipids and proteins. In this context disintegration does not mean destruction, as further described below. The multicellular organism includes lipid packing, and the causing step results in loosening of lipid packing. The multicellular organism includes membrane proteins, and the causing step results in untightening of membrane proteins in an area that includes the membrane proteins.

The multicellular organism also includes protein structure and protein function, and the causing step results in changes in the protein structure and protein function. The multicellular organism also includes membrane lipids, lipid packing, membrane proteins, protein structure and protein function, and the causing step results in temporary disintegration of the membrane lipids, loosening of the lipid packing, untightening of the membrane proteins, and changes in the protein structure and the protein function. The multicellular organism also includes cellular layers, and the temporary disintegration of membrane lipids and proteins leads to enhanced membrane permeation of nutrients and water into the cellular layers.

Another method of the invention is to promote increased cellular hydration in a multicellular organism that includes water by decreasing the density of at least some of the water in the aqueous solution.

In accord with these and other objects, the present invention provides a beverage composition comprising a carbohydrate clathrate component that includes cyclodextrins, in a concentration of 0.01-5% w/w; a complex-forming compound, in a concentration that is less than the clathrate component; an aqueous liquid component, chosen from the group consisting of still and carbonated aqueous liquids; wherein an inclusion complex is formed with at least some of the clathrate component and at least some of the complex-forming compound.

The present invention provides a beverage composition comprising cyclodextrin and complex-forming compound (also referred to as an agent). The cyclodextrin and the complex forming agent, generally, are present in a molar ratio of about 1:1. However, the invention includes mixtures of cyclodextrin and complex-forming agents in a range of molar ratios from 1:10 to 10:1 and, more narrowly, in a range of molar ratios of 1:1 to 10:1. There are two types of complex-forming compounds for purposes of this invention. The first type is simply referred to as complex-forming compounds are several non-limiting examples are given below in the Detailed Description section. A second type if “outer sphere” complexing agents, and non-limiting examples of these are also given below in the discussion of electrolytes, including both the cations and anions described in that section below. For certain complex-forming agents like arginine and niacin, the ratio could also be stated as a mass ratio, and in these cases, the mass ratio for cyclodextrin and arginine or niacin is about 10:1.

The cyclodextrin of the beverage composition is an alpha-cyclodextrin, a beta-cyclodextrin, or a gamma-cyclodextrin or combinations thereof. The complex-forming compound is selected from L-arginine, citrulline, creatine, taurine, nicotinic acid, nicotinamide, resveratrol, curcumin, thiamine, natural colorants like betalains from beetroot, flavonoids, and other compounds described below.

In an embodiment, the beverage composition comprising cyclodextrin and complex forming compound comprises: 0.05% alpha-cyclodextrin in water, 0.05% alpha-cyclodextrin-L-Arginine inclusion complex in water, 0.05% alpha-cyclodextrin-nicotinamide inclusion complex in water, 0.05% alpha-cyclodextrin-nicotinic acid (niacin) complex in water.

In another embodiment, the cyclodextrin is present in a concentration range from 0.025% to 0.1%.

In another embodiment, the present invention comprises gamma cyclodextrins based beverage compositions along with complex-forming compound.

In a still further embodiment, the present invention comprises beta-cyclodextrin-based compositions along with complex-forming compound. The composition comprises 0.01-0.05% of beta-cyclodextrin.

As to compositions and systems, the present invention also provides a beverage composition for promoting cellular hydration on ingestion by a multi-cellular organism. The present invention also provides a beverage composition that promotes increased lifespan when ingested by a multicellular organism. The present invention also provides a system that promotes cellular hydration when ingested by a multicellular organism.

In one of the embodiments, the ratio of clathrate component to complex-forming compound is in a range from about 5:1 to about 15:1.

In a another embodiment, the present invention provides a method for increasing hydration of cell system to promote cellular hydration in a multicellular organism when the mixture is ingested, the multicellular organism containing membrane lipids, lipid packing and membrane proteins, protein structure and protein function, and membrane permeation of nutrients and water, the method comprising the step of: causing the multicellular organism to ingest an aqueous solution that contains an amount of a carbohydrate clathrate component; and changing the multicellular organism by (i) temporary disintegration of the membrane lipids, (ii) loosening of the lipid packing and membrane proteins, and (iii) altering the protein structure and protein function, collectively to enhance membrane permeation of nutrients and water.

### DETAILED DESCRIPTION

Water structure is purposefully increased, or organized, by addition of one or more solutes or suitable molecular aggregates whose surfaces are capable of strongly competing with water molecules for H-bonding and/or dipole orientation. In particular, factors and agents that strengthen water molecule interactions and increase water structure thereby alter the hydration, or solvation, of a further molecular surface. Thus, a primary solution additive that increases water structure may increase hydration interactions (e.g., bonding strength and kinetics) with a molecular surface of a secondary solution component, or alternatively decrease such interactions, depending on the H-bonding surface characteristics of the secondary component.

In addition, factors that modify water structure typically change the average distance between water molecules, and may thereby increase or decrease water density. For example, as water temperature decreases below its freezing point, H-bonding between the water molecules overcomes the kinetic energy of the water molecules, resulting in an increase in water structure that decreases the density of frozen water by approximately 9%. Similarly, in liquid state water, an increase in the strength of water H-bonding increases the average distance between water molecules, which is observed as an increase in specific volume (i.e., decrease in density). A decrease in density of liquid water may increase the diffusivity of a dissolved solute. Thus, an aqueous additive component which decreases water density may increase the diffusivity of a co-dissolved solute.

Chaotropes, as used herein, are aqueous solute additives that disrupt hydrogen bonded networks in aqueous solutions, and thereby act to decrease water structure. Chaotropes typically are less polar and have weaker H-bonding potentials than water molecules. Chaotropes may preferentially bind to non-polar solutes and particles, and thereby increase solubility of a non-polar solute.

Kosmotropes, as used herein, are solutes that promote strong and extended H-bonded networks in aqueous solutions, and which thereby increase and/or stabilize the sub-micrometer scale structure of water molecule interactions. A kosmotrope having an H-bonding chemical potential greater than that of water, and/or having a dipole moment greater than that of water, may increase H-bonded networks between water molecules. Further, by strengthening hydration structure, a kosmotrope may increase hydration interactions at a molecular surface, which may include a binding site between molecules. A kosmotrope may thus be used as an aqueous solution additive to stabilize molecular interactions.

Further, a kosmotrope may increase the effective chemical activity of a dissolved co-solute. An increase in the strength of H-bonding interactions between water molecules causes water to adopt a more open architecture having a lower specific density and higher specific volume. Thus, by causing a decrease in density, addition of a kosmotrope to an aqueous solution may increase a diffusivity of one or more of a dissolved co-solute species or compounds. Increasing the diffusivity of a solute species or compound may increase its reactivity, chemical potential, effective concentration, and availability.

As discussed herein, clathrate components are amphipathic carbohydrate compounds which have external surfaces that are hydrophilic and H-bond strongly with water, and also internal surfaces that are less hydrophilic. A clathrate's internal surface may selectively bind a molecular structure which is relatively non-polar or less hydrophilic than water.

An inclusion complex, as used herein, is a chemical complex formed between two or more compounds, where a first compound (also referred to as a host) has a structure that defines a partially enclosed space into which a molecule of a second compound (also referred to as a guest) fits and binds to the first compound. The host molecule may be referred to as a clathrate and may bind the guest molecule reversibly or irreversibly.

A biological cell, as used herein, is the self-replicating functional metabolic unit of a living organism, which may live as a unicellular organism or as a sub-unit in a multicellular organism, and which comprises a lipid membrane structure containing a functional network of interacting biomolecules, such as proteins, nucleic acids, and saccharides. Biological cells include prokaryote cells, eukaryotic cells, and cells dissociated from a multicellular organism, which may include cultured cells previously derived from a multicellular organism.

A biological cell system, as used herein, is a functionally interconnected network of biological cells and/or sub-cellular elements, which may include living cells, non-living cells, cellular organelles, and/or biomolecules.

A bioactive molecule, as used herein, is a molecular compound having a functional activity in a biological cell system.

A biomolecule, as used herein, is a molecular compound that is synthesized by a biological cell. Biomolecules include compounds normally synthesized by cells, and compounds synthesized by genetically engineered cells, and chemically synthesized copies of cell-derived compounds.

A biomolecular surface, as used herein, is an outer atomic boundary of a biomolecule, which may include a biochemical interaction surface, such as a binding site.

Cellular components, as used herein, are functional elements of a biological cell, which include biomolecules, biomolecule complexes, organelles, polymeric structures, membranes and membrane-bound structures, and may further include functional pathways and/or networks, such as a sequence of molecular events.

The density of a substance is the mass per unit volume of that substance under specified conditions of temperature and pressure.

The specific volume of a substance is the volume per unit mass of the substance, which may be expressed, for example, as m³/kg. The specific volume of a substance is equivalent to the reciprocal of the density of that substance.

A biologically active component, as used herein, is a molecular substance that modifies (increases or decreases) an activity of a biological cell system.

A bioactive agent, as used herein, is a substance that when added to a biological cell system, or to a cellular component, causes a change in the biological activity of that system, or that component.

The bonded structure of water, as used herein, refers to the network of H-bonds that hold and organize the orientation of water molecules in liquid and solid states. Water structure, as used herein, increases when H-bonds between water molecules at a given temperature are strengthened, and decreases when H-bonds between water molecules at a given temperature are weakened.

An interaction between cellular components, as used herein, refers to a chemical binding between biomolecular surfaces. Such interaction may include binding between two biomolecules, such as a ligand and its specific receptor. Alternatively, such interaction may include binding between a biomolecule and an organelle, such as a cell membrane.

Extracellular signals, as used herein, are biomolecules that can modify (increase or decrease) an activity of a cell when applied to the outside of the cell. An extracellular signal may bind to a component of the cell's plasma (outer) membrane, or alternatively may pass through the plasma membrane to regulate an intracellular activity. Extracellular signals may include, but are not limited to, extracellular matrix components; cell membrane components such as glycoproteins and glyocolipids; antigens; and diffusible biomolecules such as nitric oxide.

An intracellular messenger, as used herein, is an internal component of a biological cell that has an active state, and which serves in an active state as an intermediate signal to transmit an extracellular signal to an intracellular target.

A mechanism of action, as used herein, refers to a process, which may be a step-by-step one, that takes place to achieve a certain or desired outcome.

A multi-cellular organism, as used herein, refers to an organism that consists of more than one cell, and includes organisms as complex as mammals, including animals and humans, to less complex ones such as C. elegans and other nematodes, and to as plants and other vegetation.

A pharmacological agent, as used herein, is a synthetic chemical substance that binds to and thereby alters the activity of a biomolecule or a biomolecule complex.

The present invention includes active compositions that increase an activity of a biological cell system by increasing the hydration of one or more components of that cell system.

Preferably, an active composition for modifying cellular hydration includes a primary carbohydrate clathrate component that increases the H-bonded structure of water. In some examples, the active composition preferably includes a primary carbohydrate clathrate component that increases the H-bonded structure of water and a secondary solute compound, which may be a bioactive agent. In some examples, the active composition preferably includes an inclusion complex formed between a clathrate component and a complex-forming compound, which may be a bioactive agent.

Biological cells are multi-compartment structures, comprising chemically active water-based chambers and lipid-based membranes. The structure and activity of cells derives from highly selective chemical bonding associations between their biomolecular components, such as lipids, structural proteins, enzymatic proteins, carbohydrates, salts, nucleotides, and other metabolic and signaling biomolecules. The strength and specificity of biomolecule bonding reflects complementary chemical topologies at the bonding interface. Hydrophilic and/or hydrophobic surfaces commonly dominate the chemical topology of biomolecular bond interfaces. In aqueous systems, hydrophobic and hydrophilic interactions are substantially driven by competing hydration interactions with molecules of water, whose concentration exceeds 50 M.

Cellular hydration, as used herein, refers to interaction between water molecules and biomolecular components of a cellular system. Cellular hydration may be modified by changing the strength and/or kinetics of H-bonding between water molecules and biomolecular surfaces. An aqueous solution additive that modifies water structure may, by modifying the hydration of biomolecular binding surfaces, alter the strength, kinetics, and/or specificity of binding between cellular components. For example, a kosmotrope aqueous additive that increases water structure may alter the strength, kinetics, and/or specificity of binding between a secreted intercellular signaling factor and a cognate receptor located in the plasma membrane of a potential target cell for that factor, and hence bias the outcome of a cellular signaling network.

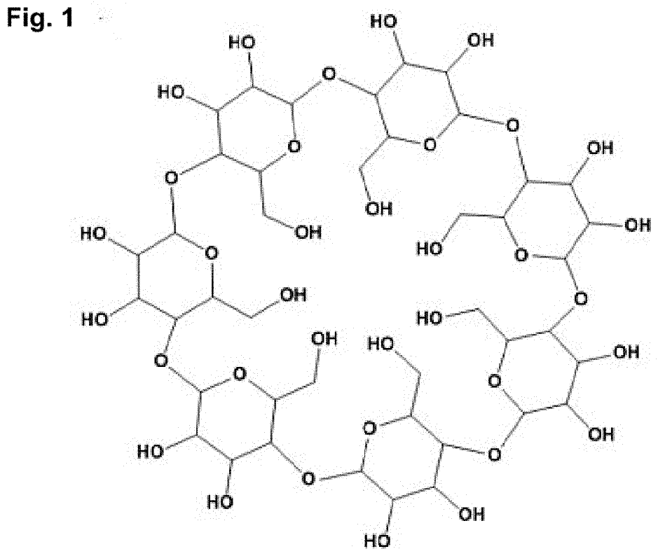

Clathrates that are suitable as active components of cellular hydration according to the present invention include amyloses and cyclodextrins. Amyloses are linear polysaccharides of D-glucose units. As shown in FIG. 1, cyclodextrins are macrocyclic oligosaccharides of D-glucose units linked by α(1-4) interglucose bonds. Amylose and cyclodextrin are readily prepared in large quantities from hydrolyzed starch. Cyclodextrin preparation includes enzymatic conversion, most commonly using the enzyme cyclodextrin-glycosyl transferase produced by Bacillus strains.

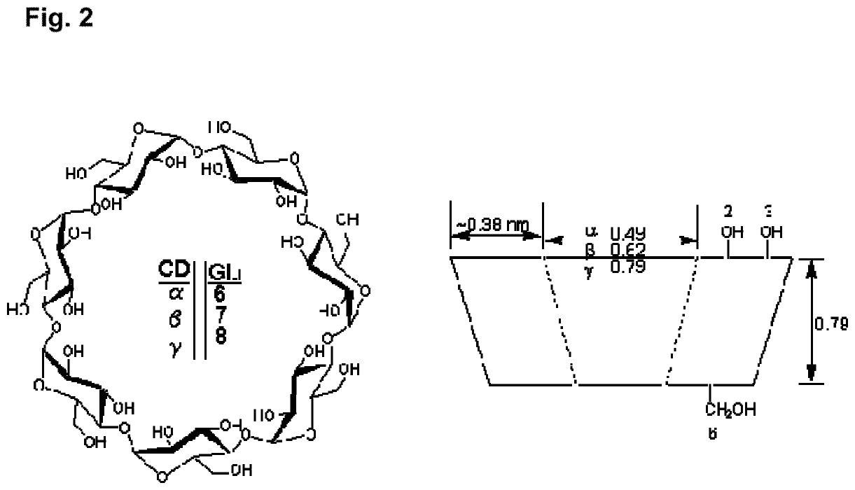

As shown in FIG. 2, cyclodextrins may differ by the number of glucose units included in the ring. Cyclodextrin species include α-cyclodextrin (6 units), β-cyclodextrin (7 units), γ-cyclodextrin (8 units), and δ-cyclodextrin (9 units). Parent cyclodextrins, as used herein, are natural, chemically underivatized α- β- and γ-cyclodextrins, having 18 (α-), 21 (β-) and 24 (γ) free, unmodified hydroxyl groups, respectively.

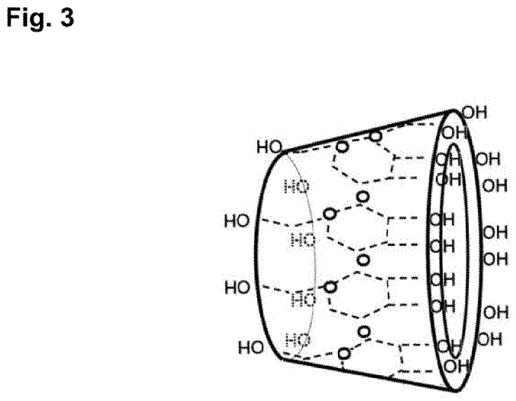

As schematically shown in FIG. 3, cyclodextrins have a toroid topology, a shape which generally resembles a truncated cone, or half of an open-ended barrel. Accordingly, a cyclodextrin may be described as including an exterior chemical surface, which includes the outer surface and the rims of the barrel, and an interior chemical surface surrounding an internal cavity (the inside of the barrel).

Cyclodextrin exterior surfaces include a high density of hydrophilic chemical groups that H-bond with water. In particular, the hydroxyl groups of the parent α-cyclodextrin, β-cyclodextrin, and γ-cyclodextrin structures are all concentrated at the ends of the cyclodextrin barrel. More particularly, cyclodextrin hydroxyl (—OH) chemical groups are located along the barrel rims, and their orientation is sterically restricted. Hydroxyl groups at glucose position C(6), which may be called primary OH groups, point in a counter-clockwise direction with respect to the narrower open end of the cyclodextrin barrel. Hydroxyl groups at glucose position C(2), which may be called secondary hydroxyl groups, angle in a clockwise direction with respect to the wider open end of the cyclodextrin barrel.

The high density and constrained orientation of cyclodextrin hydroxyl groups creates particularly strong H-bonding surfaces at both ends of the cyclodextrin barrel. Physicochemical analysis and solvation modeling of cyclodextrins show water molecules adjacent the cyclodextrin have fixed positions and low angular (rotational) mobility. Usefully, species of cyclodextrin, which differ in barrel diameter as well as number of hydroxyl groups, also differ in the number and mobility of strongly bound water molecules.

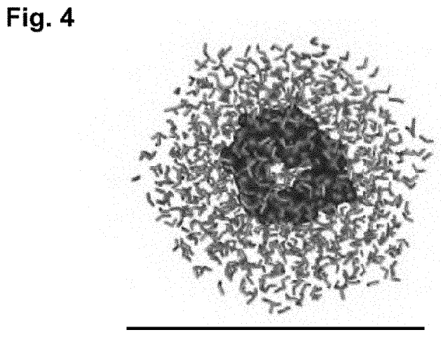

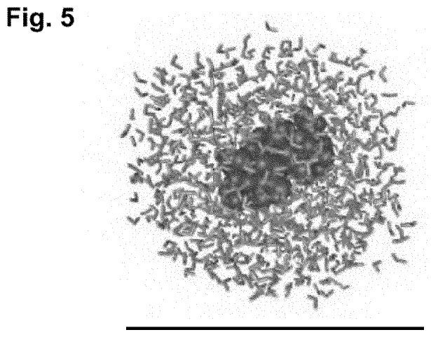

The H-bonding activity of a cyclodextrin compound may propagate into a surrounding aqueous medium. As shown in FIGS. 4 and 5, dynamic modeling of a cyclodextrin molecule introduced into a defined population of water molecules at standard temperature and pressure causes a nanosecond reorganization of water throughout the volume. FIG. 4 depicts a population distribution at one picosecond (ps) after initiating the mixing simulation; FIG. 5 depicts a redistribution of the same population at 1000 ps (1 nanosecond), wherein water molecules have adopted a more open structure.

In some examples, a cyclodextrin may function as an active component of cellular hydration through a kosmotrope activity that increases the bonded structure of water, wherein an increase in H-bonding between water molecules modifies the hydration of biomolecular surfaces, and thereby alters the strength, kinetics, and/or specificity of binding between cellular components. In some examples, a cyclodextrin may function as an active component of cellular hydration through a kosmotrope activity that increases the bonded structure of water, wherein stronger H-bonding between water molecules causes an open water structure having a lower specific density (i.e., a higher specific volume), and wherein a rate of diffusion of bioactive molecules is increased. Such examples may include a soluble bioactive molecule such as an enzyme, enzyme substrate, nutrient, metabolite, cytokine, neurotransmitter, hormone, extracellular signal, intracellular messenger, or pharmacological agent.

An active component of cellular hydration that increases a rate of diffusion in water may regulate one of the many biological processes that are limited by the rate of change in the concentration of a bioactive component. For example, clearance of a neurotransmitter from synaptic clefts is commonly diffusion limited, including the passive dispersal of glutamate from excitatory synapses in the mammalian brain, and the active catabolism of acetylcholine at vertebrate neuromuscular synapses by the diffusion-limited enzyme acetylcholine esterase. Similarly, the activity of electrically excitable cells, such as muscle cells, is commonly coordinated by the diffusion-limited changes in the concentration of the intracellular second messenger signal calcium.

The cellular hydration activity of a cyclodextrin may be modified, either increased or decreased, by forming an inclusion complex with a complex-forming compound. Internal surfaces of cyclodextrins lack hydroxyl groups, are less hydrophilic than the surrounding aqueous environment, and thereby preferentially bind co-solute molecules having low hydrophilic and H-bonding potential.

Upon ingestion by an animal, carbohydrate clathrate compositions that increase the hydrogen bonding structure of interstitial and intracellular fluids may improve cellular hydration, including hydration structure at cell membrane surfaces as well as solvation of biomolecules that sub-serve healthy cell function. Improved cellular hydration may support healthy cell function by, for example, increasing the import, export, and/or diffusivity of solutes, nutrients, waste products, cytokines, metabolites, and other molecular agents supportive of cell function, differentiation, repair, growth, and survival, and by stabilizing cellular membranes in vulnerable tissues, such as muscle and nerve.

In some examples, a carbohydrate inclusion complex ingested by an animal may increase water H-bonding structure and thereby improve cellular hydration and/or diffusivity of cellular components. In some examples, a carbohydrate inclusion complex ingested by an animal may dissociate to release a free (i.e., non-complexed) cyclodextrin clathrate component that increases water hydrogen-bonding structure and thereby improves cellular hydration and/or diffusivity of cellular components. In some examples, a carbohydrate inclusion complex may increase water structure and improve cellular hydration without dissociating. In some examples, a carbohydrate inclusion complex may dissociate into a clathrate component for increasing water structure and cellular hydration, and a complex-forming compound which may further increase water-structure and/or provide other beneficial properties, such as nutrition or flavor.

The carbohydrate clathrate compositions of the present invention may be provided in various forms, including being formed into a solid powder, tablet, capsule, caplet, granule, pellet, wafer, powder, instant drink powder, effervescent powder, or effervescent tablet. Some carbohydrate clathrate compositions may also be formed as, or incorporated into, aqueous beverages or other food products. Such carbohydrate clathrate compositions may be inclusion complexes that remain reasonably stable during storage, so that the clathrate component does not dissociate from the complex-forming compound and form a stronger complex with another compound that reduces the kosmotropic activity of the complex and thereby decrease its ability to improve cellular hydration.

The present disclosure also provides methods for improving cellular hydration in an animal, such as a human. For example, some methods may include (a) preparing a beverage with a carbohydrate clathrate component and water, or by dissolving an inclusion complex formed by a carbohydrate clathrate component and a complex-forming compound capable of dissociating from carbohydrate clathrate component under physiological conditions, and (b) having the animal orally ingest the beverage, whereupon the carbohydrate clathrate component modifies the strength, extent, and kinetics of the hydrogen bonded water structure at cellular biomolecular surfaces, and does so whether in an aqueous solution, or if it is in an inclusion complex, dissociates from the complex-forming compound.

### I. Carbohydrate Clathrate Composition

The carbohydrate clathrate component may include any suitable carbohydrate including, but not limited to, α-cyclodextrin, β-cyclodextrin, γ-cyclodextrin, methylated β-cyclodextrins, 2-hydroxypropylated β-cyclodextrins, water soluble β-cyclodextrin polymers, partially acetylated α-, β-, and γcyclodextrins, ethylated α-, β-, and β-cyclodextrins, carboxy-alkylated β-cyclodextrins, quaternary-ammonium salts of α-, β-, and γ-cyclodextrins, an amylose (e.g., an acetylated amylose), and mixtures thereof.

In preferred embodiments, the carbohydrate clathrate may be selected based upon a kosmotrope activity that increases water structure alone or in combination with other solutes. Preferred cyclodextrin kosmotropes may include α-cyclodextrin, β-cyclodextrin, γ-cyclodextrin, 2-hydroxypropyl-cyclodextrins, carboxymethylated-cyclodextrins, and quaternary-ammonium-cyclodextrins.

Cyclodextrin derivatives may include alkylated, hydroxyalkylated, alkoxyalkylated, acetylated, quaternary ammonium salts, carboxyalkylated, maltosylated, and glucosylated derivatives. Alkyl groups of cyclodextrin derivatives may be straight chain or branched, may have main chain lengths of one to three carbons, and may have a total of one to six, and preferably one to three carbon atoms. Some non-limiting examples of cyclodextrin derivatives may include methylated beta-cyclodextrins, 2-hydroxypropylated β-cyclodextrins, water soluble beta-cyclodextrin polymers, partially acetylated α-, β, and/or γ-cyclodextrins, ethylated α-, β-, and/or γ-cyclodextrins, carboxyalkylated β-cyclodextrins, quaternary ammonium salts of α-, β, and/or γ-cyclodextrins, as well as mixtures of any combination of these derivatives, together or in combination with one or more cyclodextrins. An exemplary mixture of cyclodextrins may include a combination of α-, β, and/or γ-cyclodextrin in a weight ratio range of about 1:1:1 to 2:2:1, respectively. The cyclodextrin may be in a hydrate crystalline and/or amorphous form, including but not limited to the hydrate and/or amorphous forms of α-, β, and/or γ-cyclodextrin, and mixtures thereof.

If the carbohydrate clathrate composition is in solid form, the cyclodextrin component may be present in a concentration range of about 10-90% w/w, or about 15-70% w/w, or about 15-60% w/w. Preferably, the cyclodextrin component may be present in a concentration range of about 10-50% w/w, or about 15-40% w/w. More preferably, the cyclodextrin component may be present in a concentration range of about 20-25% w/w.

If the carbohydrate clathrate composition is in the form of an aqueous beverage, the cyclodextrin component may be present in a concentration range of about 0.01-75% w/w, or about 0.05-50% w/w, or about 0.1-25% w/w. Preferably, the cyclodextrin component may be present in a concentration range of about 0.1-10% w/w. More preferably, the cyclodextrin component may be present in a concentration range of 0.1-5% w/w.

The carbohydrate clathrate composition may preferably include a clathrate capable of forming an inclusion complex with a variety of complex-forming compounds, such as amino acids, vitamins, flavorants, odorants, colorants, and the like. Non-exclusive examples of carbohydrate clathrate components capable of binding a complex-forming compound to form an inclusion may include α-cyclodextrin, β-cyclodextrin, γ-cyclodextrin, 2-hydroxypropyl-cyclodextrins, carboxymethylated-cyclodextrins, quaternary-ammonium-cyclodextrins, amyloses, amylose derivatives, or any desired mixture of these.

A cyclodextrin clathrate component may be further selected based upon its desired binding properties with selected complex-forming compounds. Non-limiting examples of acceptable cyclodextrins may include commercially available and government regulatory approved forms of α-, β- and γ-cyclodextrins. The number of glucose units determines the internal dimensions of the cavity and its volume, and may determine a selectivity in forming inclusion complexes with a guest molecule. Selected complex-forming compounds, when bound to a host cyclodextrin or other host carbohydrate clathrate, may modify the physico-chemical properties of the complexed host to increase its kosmotropic activity.

If the clathrate component is in the form of an amylose component, the amylose component may contain glucose units expressed as degree of polymerization (DP) in the range of DP=10-900, and more preferably DP=20-200, and most preferably DP=30-80. Amylose derivatives may include, but are not limited to, acetylated amyloses. The amylose component preferably may have a structure that includes α1,4-linked D-glucopyranoses in a helical arrangement that defines a central cavity for binding hydrophobic molecules. For example, the A- and B-starch helix of V-amylose may include a parallel, left-handed double helix defining a central cavity. The helices of amylose inclusion complexes may be stabilized by the hydrophobic forces created by the host-guest interactions, intermolecular H-bonds between glucoses in adjacent amyloses, and intramolecular H-bonds formed by adjacent turns of the helix. See Hinrichs, W., et al., “An Amylose Antiparallel Double Helix at Atomic Resolution,” Science, (1987), 238(4824): 205-208, the complete disclosure of which is hereby incorporated by reference for all purposes. An amylose clathrate component maybe used to form an inclusion complex with a complex-forming compound having a low molecular weight, such as the non-limiting examples of flavorants, colorants, vitamins, amino acids, and/or amines.

If the composition containing an amylose clathrate component is in solid form, the amylose component preferably may be present in a concentration range of about 10-90% w/w, or about 15-70% w/w, or about 15-60% w/w. More preferably, the amylose component may be present in a concentration range of about 10-50% w/w, or about 15-40% w/w. Most preferably, the amylose component may be present in a concentration range of about 20-25% w/w. If the composition containing the amylose clathrate component is in the form of an aqueous beverage, the amylose component preferably may be present in a concentration range of about 0.1-75% w/w, or about 1-50% w/w, or about 1-25% w/w.

### II. Complex-Forming Compound

In some examples, the clathrate compositions disclosed herein may optionally contain a complex-forming compound (also referred to as an agent), which may include one or more amino acids, vitamins, flavorants, odorants, and/or other nutritional components, as well as combinations or mixtures of these agents. The carbohydrate clathrate compositions may further include one or more carbonation forming components for use in forming beverage products.

The complex-forming compound may strongly complex with the clathrate component so as to increase a kosmotropic activity and thereby influence cellular hydration. Alternatively, these agents may weakly complex with the clathrate component so as to have the capability of dissociating therefrom in order to allow a free clathrate component to increase water structure.

As used herein, it is intended that a complex-forming compound is any compound that has utility in the beverage compositions described below, regardless of how strong or weakly it complexes with the clathrate component, and even if it does not complex at all with the clathrate component. As noted above, there are two types of complex-forming compounds, a first type being simply referred to as complex-forming compounds, and a second type of “outer sphere” complexing agents.

Non-limiting examples of amino acids suitable for forming inclusion complexes with the carbohydrate clathrate compositions of the present disclosure may include aspartic acid, arginine, glycine, glutamic acid, proline, threonine, theanine, cysteine, cystine, alanine, valine, tyrosine, leucine, isoleucine, asparagine, serine, lysine, histidine, ornithine, methionine, carnitine, aminobutyric acid (alpha-, beta-, and gamma-isomers), glutamine, hydroxyproline, taurine, norvaline, sarcosine, salts thereof, and mixtures thereof. Also included are N-alkyl C₁-C₃ and N-acylated C₁-C₃ derivatives of these amino acids, and mixtures of any of the amino acids or derivatives thereof. Preferred complex forming amino-acids that may be included with cyclodextrins to increase water structure and cellular hydration include L-arginine, L-lysine, N-methyl-lysine, and L-carnitine.

Non-limiting examples of vitamins may include nicotinamide (vitamin B₃), niacinamide, niacin, pyridoxal hydrochloride (vitamin B₆), ascorbic acid, edible ascorbyl esters, riboflavin, pyridoxine, thiamine, vitamin B₉, folic acid, folate, pteroyl-L-glutamic acid, pteroyl-L-glutamate, salts thereof, and mixtures thereof. Preferred vitamins included with cyclodextrins to increase water structure and cellular hydration may include nicotinamide and niacinamide.

Non-limiting examples of flavorants may include apple, apricot, banana, grape, blackcurrant, raspberry, peach, pear, pineapple, plum, orange, and vanilla flavorants. Examples of flavorant related compounds include butyl acetate, butyl isovalerate, allyl butyrate, amyl valerate, ethyl acetate, ethyl valerate, amyl acetate, maltol, isoamyl acetate, ethyl maltol, isomaltol, diacetyl, ethyl propionate, methyl anthranilate, methyl butyrate, pentyl butyrate, and pentyl pentanoate. A flavorant may be selected so that it weakly binds to a selected cyclodextrin component with a binding constant in the range of about 10 to 800 M⁻¹, preferably 30 to 150 M⁻¹, and more preferably 40 to 100 M⁻¹.

Non-limiting examples of other taste improving components may include polyol additives such as erythritol, maltitol, mannitol, sorbitol, lactitol, xylitol, inositol, isomalt, propylene glycol, glycerol (glycerine), threitol, galactitol, palatinose, reduced isomalto-oligosaccharides, reduced xylo-oligosaccharides, reduced gentio-oligosaccharides, reduced maltose syrup, and reduced glucose syrup.

Non-limiting examples of colorants may include those that are known to be more water soluble and less lipophilic. Examples of colorants with those properties are betalains, which may be from beetroot. Examples of betalains include betacyanins and betaxanthins, including vulgaxanthin, miraxanthin, portulaxanthin and indicaxanthin; anthocyanidins, such as aurantinidin, cyanidin, delphinidin, europinidin, luteolinidin, pelargonidin, malvidin, peonidin, petunidin and rosinidin, as well as all corresponding anthocyanins (or glucosides) of these anthocyanidins; and turmeric type colorants including phenolic curcuminoids, such as curcumin, demethoxycurcumin and bisdemethoxycurcumin.

In addition to those described above, non-limiting examples of other complex-forming compounds may include curcumin, polyphenols, dihydrocurcumin, spermidin, L-lysin, reservatrol, coenzymeQ10, delta-tocopherol, delphindin, caffeine, and guarna.

Another group of non-limiting examples of complex-forming compounds, of the outer-sphere type, are electrolytes, and specifically, magnesium, sodium, potassium, chloride, calcium, phosphate, and bicarbonate.

All of the above examples of amino acids, vitamins, flavorants and related compounds may be in appropriate salt or hydrate forms.

The complex-forming compound may be selected to form an inclusion complex with a selected clathrate component. The complex-forming compound may bind to the clathrate component as a guest molecule in the cavity of the clathrate molecule, and/or may form a so-called outer sphere complex, where the selected weak complex-forming compound binds to the clathrate molecule at a position at or around the rim(s) of the clathrate. For example, the selected weak complex-forming compound may be bound to a cyclodextrin molecule at or around the primary and/or secondary hydroxyl groups at the rims of the cyclodextrin torus. Some complex-forming compound that form an outer sphere complex with the selected cyclodextrin may reduce or prevent self-aggregation of dissolved, hydrated cyclodextrin molecules by masking intermolecular hydrogen bonds that form between two neighboring cyclodextrin molecules in water.

If the carbohydrate clathrate composition is in solid form, the complex-forming compound may be present in a concentration range of about 1-50% w/w. Preferably, the complex-forming compound may be present in a concentration range of about 1-40% w/w or about 1-25% w/w. More preferably, the complex-forming compound may be present in a concentration range of about 5-15% w/w.

If the carbohydrate clathrate composition is in the form of an aqueous beverage, the complex-forming compound may be present in a concentration range of about 0.1-25% w/w or about 1-20% w/w. Preferably, the complex-forming compound may be present in a concentration range of about 1-15% w/w or about 1-10% w/w or about 3-8% w/w. More preferably, the complex-forming compound may be present in a concentration range of about 5-8% w/w.

### III. The Inclusion Complex

As noted above, the inclusion complex may include a clathrate host molecule complexed with one or more complex-forming compound. In the form of a solid product, such as a solid powder or tablet, the inclusion complex may exhibit some unique properties as compared to a solid composition containing essentially the same components, but without the preliminary formation of the inclusion complex. The inclusion complex is essentially a chemical entity having non-covalent hydrogen bonds formed between the clathrate molecule and the weak complex-forming compound molecule. The inclusion complex, in its solid form, has the potential of dissociating into the clathrate component for increasing water structure and the complex-forming compound, which may further increase water structure or provide other beneficial properties, such as nutrition or flavor, when the inclusion complex is introduced to an aqueous environment, such as upon dissolution in an aqueous beverage, or upon ingestion.

When in the form of a solid product, the clathrate component and one or more types of a complex-forming compound may be substantially in the form of an inclusion complex, as described above. Preferably, over about 25% of the clathrate component is complexed with one or more types of a complex-forming compound in the form of an inclusion complex. It is progressively more preferable to have over 35%, 45%, 50%, 60%, 70%, 80%, 90%, and 95% of the clathrate component complexed.

### IV. Carbonation-Forming Components

Some clathrate compositions may include carbonation-forming components that produce carbonation, or effervescence, upon dissolution into an aqueous environment. Carbonation-forming components advantageously may inhibit self-aggregation of clathrate molecules, thereby increasing clathrate surface area for structuring water and increasing cellular hydration. Non-limiting examples of carbonation-forming components may include sodium carbonate, sodium bicarbonate, potassium carbonate and potassium bicarbonate. Preferred carbonation-forming components may include sodium carbonate, and sodium bicarbonate.

If the carbohydrate clathrate composition is in solid form, the carbonation-forming component may be present in a concentration range of about 1-60% w/w or about 5-60% w/w. Preferably, the carbonation-forming component may be present in a concentration range of about 5-45% w/w or 10-45% w/w. More preferably, the carbonation-forming component may be present in a concentration range of about 10-15% w/w.

If the carbohydrate clathrate composition is in the form of an aqueous beverage, the carbonation-forming component may be present in a concentration range of about 1-30% w/w or about 1-25% w/w. Preferably, the carbonation-forming component may be present in a concentration range of about 2-15% w/w or 2-10% w/w. More preferably, the carbonation-forming component may be present in a concentration range of about 2-5% w/w.

### V. Other Components

Some compositions may include yet other components that affect the taste and/or nutritional value of the composition. These additional components may include, but are not limited to, one or more of the following: flavor additives, nutritional ingredients and/or various hydroxyl-acids that act as clathrate aggregation-preventing additives in the formulations. Non-limiting examples of such other components may include citric acid, ascorbic acid, sodium chloride, potassium chloride, sodium sulfate, potassium citrate, magnesium sulfate, alum, magnesium chloride, maltodextrin, mono-, di-, tri-basic sodium or potassium salts of phosphoric acid (e.g., inorganic phosphates), salts of hydrochloric acid (e.g., inorganic chlorides), sodium bisulfate. Non-limiting examples of hydroxyl-acids that prevent cyclodextrin aggregation may include isocitric acid, citric acid, tartaric acid, malic acid, threonic acid, salts thereof and mixtures thereof. These hydroxyl-acids also may exhibit some nutritional benefits. Other non-limiting examples of additional optional components, such as taste additives, that may be used include suitable organic salts, such as choline chloride, alginic acid sodium salt (sodium alginate), glucoheptonic acid sodium salt, gluconic acid sodium salt (sodium gluconate), gluconic acid potassium salt (potassium gluconate), guanidine HCl, glucosamine HCl, amiloride HCl, monosodium glutamate (MSG), adenosine monophosphate salt, magnesium gluconate, potassium tartrate (monohydrate), and sodium tartrate (dihydrate).

Preferred other components may include, for example, citric acid, ascorbic acid, and maltodextrin.

If the carbohydrate clathrate composition is in solid form, the one or more other components each may be present in a concentration range of about 1-30% w/w or about 1-25% w/w. Preferably, the one or more other components each may be present in a concentration range of about 1-20% w/w or 1-15% w/w. More preferably, the one or more other components each may be present in a concentration range of about 2-5% w/w.

If the carbohydrate clathrate composition is in the form of an aqueous beverage, the one or more other components may be present in a concentration range of about 1-20% w/w or about 1-15% w/w. Preferably, the one or more other components may be present in a concentration range of about 1-10% w/w or 1-5% w/w. More preferably, the one or more other components may be present in a concentration range of about 1-3% w/w.

### VI. Component Ratios

In addition to the above descriptions regarding the types and amounts of the various components that may be employed in the carbohydrate clathrate compositions disclosed herein, it is additionally noted that the relative amounts of these components can be described as well. Preferably, the weight ratio of the clathrate component to the complex-forming compound may be in the range of about 5:1 to 1:10, more preferably may be in the range of about 2:1 to 1:5, still more preferably may be in the range of about 2:1 to 1:2, and yet more preferably may be in the range of about 1:1 to 1:2.

Regarding the other possible components, such as flavor components, carbonation-forming components, and other components described above, the weight ratio of the clathrate component to each of the other components separately may be in the range of about 25:1 to 1:25, or about 10:1 to 1:10, or about 5:1 to 1:5, or optionally about 2:1 to 1:2, as well as 1:1.

The present invention provides a beverage composition, system and method of use, and a mechanism of action of the beverage composition for increasing cellular hydration and for increasing lifespan.

In an embodiment, the present invention provides a beverage composition comprising a carbohydrate clathrate component that includes cyclodextrin, in a concentration of 0.01-5% w/w; a complex-forming compound; an aqueous liquid component, chosen from the group consisting of still and carbonated aqueous liquids; wherein an inclusion complex is formed with at least some of the clathrate component and at least some of the complex-forming compound.

Further, the ratio of clathrate component to complex-forming compound is preferred in the range from about 5:1 to about 15:1.

In another embodiment, the beverage composition of the present invention comprises a cyclodextrin, or mixture of cyclodextrins, and complex forming compound. One embodiment may include 0.05% alpha-cyclodextrin in water, 0.05% alpha-cyclodextrin-L-Arginine inclusion complex in water, 0.05% alpha-cyclodextrin-nicotinamide inclusion complex in water, 0.05% alpha-cyclodextrin-nicotinic acid (niacin) complex in water; or mixtures of one or more of the aforementioned substances.

In another embodiment, the present invention includes gamma cyclodextrins-based beverage compositions and a the complex-forming compound.

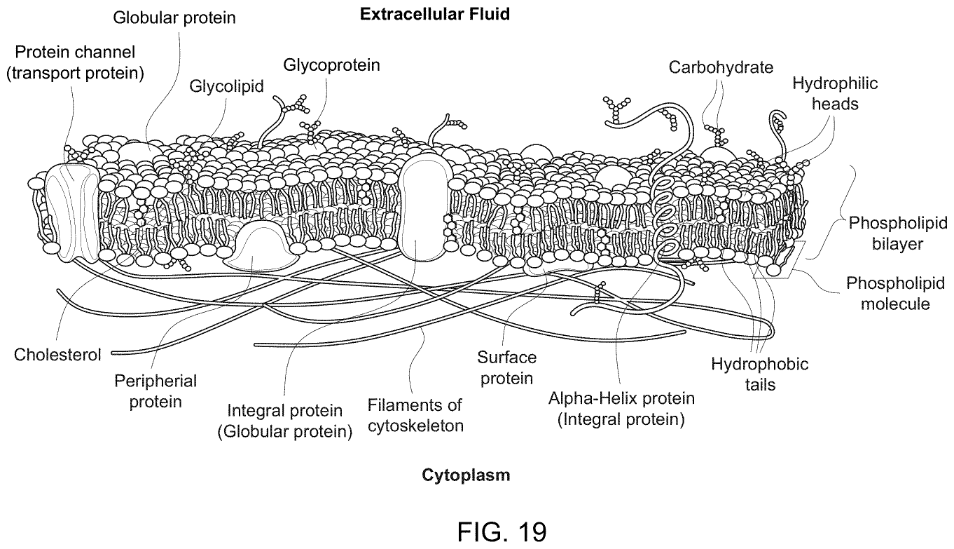

To describe the mechanism of action of the invention, certain tissue present in multicellular organisms must first be described to provide perspective of how that mechanism of action functions. A lipid bilayer or phospholipid bilayer is a thin polar membrane made of two layers of lipid molecules. The lipid bilayer is the barrier that keeps ions, proteins and other molecules where they are needed and prevents them from diffusing into areas where they should not be. Biological bilayers are usually composed of amphiphilic phospholipids that have a hydrophilic phosphate head and a hydrophobic tail consisting of two fatty acid chains. Apart from phospholipids, the bilayer comprises cholesterol which helps strengthen the bilayer and decreasing its permeability. It also comprises integral membrane proteins and other functional proteins like ion-channels, aquaporins etc. (as shown in FIG. 19).

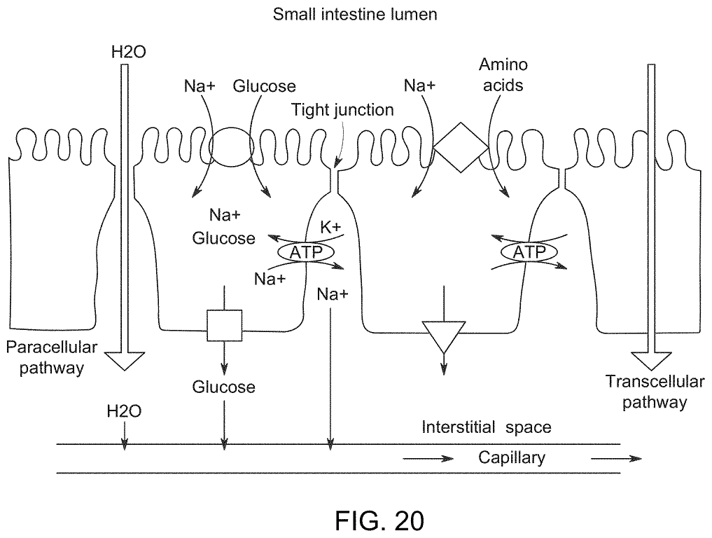

Aquaporins are the only known water channel, however, water also diffuses via passive diffusion in response to osmotic gradient established by sodium in the intestinum. The bulk of the water absorption is a transcellular process, i.e., it goes through membrane bilayers by passive diffusion via the water channels (aquaporins), but some also diffuses through the tight junctions (called paracellular pathway, and shown in FIG. 20).

According to the present invention, the cyclodextrin-based beverages influence the cellular hydration with a mechanism of temporary and reversibly changing the cell membrane lipid packing and the membrane fluidity, due to the non-covalent inclusion complex formation. This feature of the mechanism of action is also referred to as reversibly and temporarily disintegrating the membrane lipids, but disintegrating is not used in its usual sense to mean destruction. Rather, the lipids are changed or moved but the process is reversible so they can return to the location they were and can again pack together. The cyclodextrin-based beverages of the invention may comprise alpha cyclodextrin or its derivatives, or beta cyclodextrin or its derivatives.

The alpha cyclodextrin and its derivatives primarily affect the phospholipid constituents, and the membrane anchored-proteins in the vicinity of these constituents. On the other hand, beta-cyclodextrins and derivatives target mainly cholesterol and cholesterol-phospholipid complexes in the membrane.

Further, the alpha-cyclodextrin and its derivatives preferably interact with slim membrane lipid components such as glycosphingolipids, sphingomyelin, phosphatidylcholine, phosphatidyl ethanolamine etc., wherein, all these phospholipids are integral constituents of lipid rafts, where most of the membrane-bound functional proteins are located, such as ion channels, or water channels/aquaporins.

The interaction between cyclodextrins and phospholipids is a reversible non-covalent complex formation, a molecular event, during which the lipid environment of membrane-anchored proteins will alter and the cell-physiological functions of these transporter proteins (e.g. ion transport) will change leading to enhanced water transport

The lipid-cyclodextrin interaction is completely reversible that leads to change in lipid packing in a cyclodextrin concentration dependent manner. The low concentration of hydration-enhancing cyclodextrins exerts no irreversible cellular damage.

The highly hydrated form is a dissolved alpha-cyclodextrin; and in an alpha-CD water solution, water structure (monomers- and clusters) will be changed. In carbonated water, more importantly dissolved alpha-CD will contain less aggregates of the cyclodextrins and more hydrated monomers. The lower the number of aggregated alpha-CDs in water solution, the higher the number of accessible cyclodextrin cavities, ready for complex formation.

Similarly, the beta-cyclodextrin and its derivatives affect the membrane cholesterol-rich domains around the membrane-bound proteins. Both alpha- and beta-cyclodextrins cause change in membrane transport processes, initiate cell signaling, and affect water transport across aquaporins.

The cellular hydration plays a role in different cell functions and enhancement of cell hydration has an effect on cellular autophagosome formation or on autophagy. (References: S. Vom Dahl, et al. Biochem. J. 2001. 354. (1) 31-36. and Schliess, F. et al Acta Physiologic 187. 1-2. 2006, and Haussinger, D.). Further, cellular hydration state is an important determinant of protein catabolism in health and disease (Lancet 341. 8856. 1330-1332. 1993).

In another embodiment, the present invention includes a beverage composition that causes cellular hydration in a multicellular organism when a multicellular organism ingests it. The multicellular organism is capable of intracellular water permeation, and the ingestion of the composition by the multicellular organism enhances the intracellular permeation. The organism contains aquaporins, and the cellular hydration is caused by interaction of the composition with the aquaporins.

The present invention also provides a method of promoting increased cellular hydration in a multicellular organism that is capable of intracellular water permeation, comprising; causing the multicellular organism to ingest an aqueous solution that contains an amount of a carbohydrate clathrate component; and enhancing the intracellular permeation. The multicellular organism contains aquaporins and causes interaction of the composition with the aquaporins. The cyclodextrin-assisted enhancement of intracellular water permeation was assessed and corroborated by single cell Xenopus laevis frog oocytes having expressed human aquaporin AQP-1 water channels. The results of the biological tests are illustrated Example 7.

In another embodiment, the present invention provides a method of promoting increased cellular hydration in a multicellular organism that includes water and a carbohydrate clathrate component, and functions to decrease the density of at least some of the water in the aqueous solution. The physico-chemical properties for this embodiment of the invention that lowers the density of water in the aqueous solution is shown in Example 2.

Further, as described in (M. F. Chaplin Biophysical Chemistry 83 (1999) 211-221), dodecahedral water clusters have been reported at hydrophobic and protein surfaces, where low-density water with stronger hydrogen bonds and lower entropy has been found. Similar cavities have been found in low density amorphous ice (LDA) and shown to be formed relatively easily in water during molecular simulations. The basis of the model described herein is a network that can convert between lower and higher density forms without breaking hydrogen bonds. It contains a mixture of hexamer and pentamer substructures and contains cavities capable of enclosing small solutes.

The above embodiment of the invention applies this theory and results in a novel mechanism that changes the structure of water by reducing its density.

The present invention provides another method for increasing hydration of cell system to promote cellular hydration in a multicellular organism when the mixture is ingested. The multicellular organism contains membrane lipids, lipid packing and membrane proteins, protein structure and protein function, and membrane permeation of nutrients and water. The method further includes the steps of causing the multicellular organism to ingest an aqueous solution that contains an amount of a carbohydrate clathrate component; and changing the lipid bilayer structure of multicellular organism by (i) temporary disintegration of the membrane lipids, (ii) loosening of the lipid packing and membrane proteins, and (iii) altering the protein structure and protein function, collectively to enhance membrane permeation of nutrients and water.

### VII. Preferred Embodiments

Preferred embodiments of the carbohydrate clathrate composition disclosed herein are provided as illustrations, and are not intended to limit the scope of this disclosure in any way.

### Example 1

### Effect of Cyclodextrin on Molecular Dynamics of Water Structure

A simulated water solvated cyclodextrin molecular system was created using HyperChem® 5.11 software (from HyperCube Inc, Gainesville, Fla.), with input parameters derived from single crystal analysis of cyclohepta-amylose dodecahydrate clathrate (or, β-cyclodextrin) reported by Lindner and Saenger (see: Carbohydr. Res., 99:103, 1982), and using a water periodic solvent box (3.1×3.1×3.1 nm³) containing altogether 984 water molecules. Molecule conversion and atom type were adjusted to the proper format using TinkerFFE 4.2 (TINKER Software Tools for Molecular Design, Version 5.0, Jay William Ponder, Washington University, St. Louis, Mo.). Molecular mechanics and dynamics calculations were performed with Tinker 5.0 software after preliminary optimization of the truncated Newton-Raphson method using a Linux x86-64 operating system (Slamd 64 v12.2).

Molecular dynamics simulations were run using MM3 Force Field molecular mechanics software, at constant temperature (298 K) for 120 picosecond (psec), with 0.1 femtosecond (fsec) steps. Recordings were generated by dumping intermediate structures every 100,000 steps (equivalent to 10 psec elapsed time).

### Observations:

At time zero of each simulation, the standard water solvent box contained one β-cyclodextrin clathrate molecule and a uniformly distributed population of 984 water molecules. FIGS. 4 and 5 show a representation of the central portion of the solvent box at particular elapsed times during one representative simulation. It will be appreciated that water molecule positions and orientations are represented as (bent) rods, while β-cyclodextrin is represented as a van der Waals surface. It will be further appreciated that FIGS. 4 and 5 depict a volume of the solvent box, and therefore compress a three dimensional molecular distribution into two dimensions. FIG. 4 shows a central portion of the solvent box at 1 psec of elapsed time of a simulation. In particular, at 1 psec of elapsed time, water molecules immediately adjacent to β-cyclodextrin have acquired relatively static (stable) positions through H-bonding to cyclodextrin. Such water molecules may be referred to as a first hydration layer. However, the distribution of most water molecules in the solvent box remains generally similar to the starting distribution (1 psec previous), which is unstructured.

FIG. 5 shows the simulation of after 1000 psec (i.e., 1 nsec) of elapsed time. At 1000 psec, water molecules immediately adjacent to β-cyclodextrin continue to occupy relatively static (stable) positions. However, compared to 1 psec (FIG. 4), water molecules beyond the first hydration layer have acquired a more open microstructure.

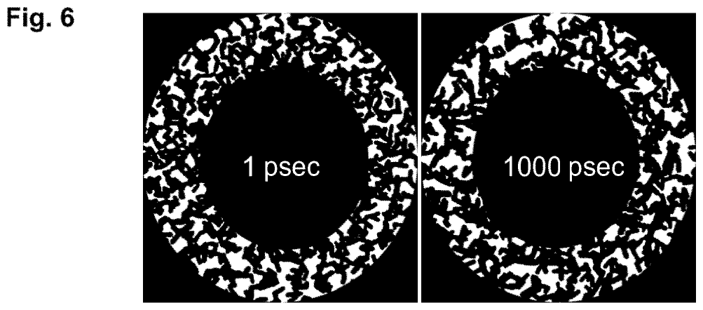

Differences in water structure may be more readily observed in the absence of the perspective shadowing detail included in FIGS. 4 and 5. FIG. 6 shows alternative views of the water molecule distributions shown in FIG. 4 (left side, labeled 1 psec) and FIG. 5 (right side, labeled 1000 psec), which were produced by the following methods: image files for FIGS. 4 and 5, having 256 grey levels (8 bits), were opened in Photoshop 9.0 (Adobe, Inc), adjusted to 300 dpi, thresholded at grey level 207; images were cropped to an identical outer annulus diameter using the circle select tool, and the outer square corners filled with black (grey level 0), and then further cropped to blacken an inner annulus that barely includes the cyclodextrin molecule. The dimensions of the outer and inner annuli are identically applied to the compared images. The resulting thresholded representations qualitatively show water molecules surrounding the central (occluded) cyclodextrin molecule have a more open and coordinated structure at 1000 psec (e.g., right side panel of FIG. 6).

To quantitatively assess the change in microstructure of water represented in FIGS. 4-6, molecular density was approximated by measuring open paths through the depicted volume, a method similar to a mean free path analysis, where a mean free path in a defined volume of a molecular substance is inversely related to the density of the molecules. In particular, an open path between water molecules is shown by a white pixel element, and the number of open paths in the volume is readily quantitated using the histogram tool of Photoshop 9.0 to count the number of white pixel elements. Applied to the panels of FIG. 6, a measured increase of 2% was calculated for open paths at 1000 psec of elapsed time compared to open paths at 1 psec of elapsed time. For comparison, freezing of pure water results in a 9% decrease in density. As path length is inversely proportional to molecule density, the analysis indicates that dissolved cyclodextrins decrease the density of an aqueous solution by increasing the organization of water molecules.

In summary, the results indicate a rapid (psec) H-bonding adhesion between the outer surface hydroxyls of β-cyclodextrin and water molecules is followed by a slower (nanosecond) propagation of water molecule reorientation throughout the solvent box, resulting in a more open water structure. The measured results further indicate that a cyclodextrin may sufficiently increase H-bonding between water molecules in the surrounding aqueous volume to result in a decrease in the density of water.

### Example 2

### Physicochemical Properties (Density Measurements)

The current study further manifests the density measurements in a cyclodextrin concentration dependent manner. The materials used were specified as: α-cyclodextrin (Wacker—food grade, internal ID: B002/18); three weak complex forming additives (in α-cyclodextrin complex, 1:1 mol/mol) are L-arginine (Sigma-Aldrich Cat. No A5006), Nicotinic acid (Sigma-Aldrich Cat. No 72309), Nicotinamide (Sigma-Aldrich Cat. No 72340) γ-cyclodextrin (Wacker—food grade, internal ID: B064/18).

Water samples used were bottled water and tap water; wherein the tap water comprises the following impurities and properties: Free active chlorine (0.18 mg/l), Chloride (24 mg/l), Iron (6 μg/l), Manganese (2 μg/l), Nitrate (9 mg/l), Nitrite (<0.03 mg/l), Ammonium (<0.04 mg/l), Hardness of water (122 mg/l CaO), Conductivity (442 μS/cm) and pH 8. Purified water was produced by removal of dissolved ions by Merck/Millipore Synergy® Water Purification System at Cyclolab. The water quality produced Type 1 water (18.2 MΩ·cm at 25° C. ultrapure water) from pretreated water.

1:1 mol/mol stoichiometry complexes were prepared for the experiment. Nicotinic acid/alpha-CD complex was prepared by dissolving 11.22 g nicotinic acid and 98.63 g alpha-CD (89.66 g on dry basis) in 700 ml purified water. Nicotinamide/alpha-CD complex was prepared by dissolving 5.57 g nicotinamide and 49.3 g alpha-CD (44.4 g on dry basis) in 350 ml purified water and L-arginine/alpha-CD complex was prepared by dissolving 15.18 g L-arginine and 94.26 g alpha-CD (85.69 g on dry basis) in 700 ml purified water. Further, for all the three complexes, the liquid was frozen in dry ice bath and lyophilized. The dry lyophilizate was ground and sieved. Clarity, pH, conductivity, density, viscosity, turbidity, surface tension and osmolality were determined in solutions prepared with purified water. The concentration noted for the complex solutions are indicating the actual alpha cyclodextrin content. Alpha & gamma cyclodextrin mix is a 50-50 weight % mixture of the two constituents and the percentage indicates the total cyclodextrin content. Tables 1-3 summarize the results of the physico-chemical tests.

TABLE 1
Physico-Chemical properties of test solutions containing alpha-and gamma cyclodextrin vs. control purified water

| | No Additive | Alpha Cyclodextrin | | | | Gamma Cyclodextrin | | | |
|---|---|---|---|---|---|---|---|---|---|
| Purified Water | 0.00% | 0.05% | 0.10% | 1.00% | 2.00% | 0.05% | 0.10% | 1.00% | 2.00% |
| pH | 6.28 | 6.20 | 6.36 | 6.55 | 6.70 | 6.82 | 6.79 | 6.87 | 6.77 |
| Conductivity | 34.3 | 35.3 | 39.0 | 39.6 | 38.7 | 25.0 | 25.0 | 27.0 | 27.0 |
| (uS · cm⁻¹) | | | | | | | | | |
| Density (at 22 C.) | 0.9980 ± 0.0005 | 0.990 ± 0.002 | 0.989 | 0.993 | 1.048 | 0.995 ± 0.001 | 0.997 | 0.999 | 1.003 |
| (g · cm⁻¹) | | | | | | | | | |
| Viscosity (25 C.) | 0.91 | 0.91 | 0.92 | 0.97 | 1.01 | .92 | .92 | .94 | 0.97 |
| (cP) | | | | | | | | | |
| Turbidity | (reference) | 0.003 | 0.000 | 0.017 | 0.015 | 0.005 | 0.008 | 0.065 | 0.108 |
| (Abs, λ = 410 nm) | | | | | | | | | |
| Visual Inspection | clear | clear | clear | clear | clear | clear | clear | hazy | hazy |
| Surface Tension | 72 | 72 | 72 | 72 | 73 | 72 | 72 | 73 | 73 |
| (mN · m⁻¹) | | | | | | | | | |
| Osmolality | 0 | 0 | 2 | 8 | 18 | 0 | 0 | 4 | 12 |

(mOsm/kg)

TABLE 2
Physico-Chemical properties of test solutions prepared of alpha-cyclodextrin complexes vs. control purified water

| | No Additive | Alpha Cyclodextrin | | | | Gamma Cyclodextrin | | | |
|---|---|---|---|---|---|---|---|---|---|
| Purified Water | 0.00% | 0.05% | 0.10% | 1.00% | 2.00% | 0.05% | 0.10% | 1.00% | 2.00% |
| pH | 6.28 | 8.88 | 9.18 | 9.87 | 10.12 | 6.48 | 6.42 | 6.55 | 6.59 |
| Conductivity | 34.3 | 46.6 | 52.6 | 97.3 | 130.0 | 40.7 | 43.6 | 44.0 | 45.8 |
| (uS · cm⁻¹) | | | | | | | | | |
| Density (at 22 C.) | 0.9980 ± 0.0005 | 0.995 ± 0.001 | 0.981 | 0.990 | 1.010 | 0.995 ± 0.001 | 0.996 | 1.003 | 1.005 |
| (g · cm⁻¹) | | | | | | | | | |
| Viscosity (25 C.) | 0.91 | 0.94 | 0.91 | 1.02 | 1.06 | 0.93 | 0.95 | 1.00 | 1.06 |
| (cP) | | | | | | | | | |
| Turbidity | (reference) | 0.006 | 0.010 | 0.095 | 0.165 | 0.0152 | 0.0296 | 0.0463 | 0.0981 |
| (Abs, λ = 410 nm) | | | | | | | | | |
| Visual Inspection | clear | clear | clear | hazy | hazy | clear | clear | hazy | hazy |
| Surface Tension | 72 | 72 | 72 | 72 | 73 | 72 | 72 | 72 | 73 |
| (mN · m⁻¹) | | | | | | | | | |
| Osmolality | 0 | 0 | 1 | 17 | 34 | 2 | 2 | 21 | 39 |

(mOsm/kg)

In Table 2, the second-fifth columns correspond to mixtures of alpha cyclodextrin with an L-arginine complex and ACD/Nicotinamide, and the sixth-ninth columns correspond to gamma cyclodextrin mixtures.

TABLE 3
Physico-Chemical properties of test solutions prepared of alpha-cyclodextrin/nicotinic acid
complex, alpha & gamma cyclodextrin mix vs. control purified water

| | No Additive | Alpha Cyclodextrin/Nicotinic acid | | | | Alpha & Gamma Cyclodextrin Mix | | | |
|---|---|---|---|---|---|---|---|---|---|
| Purified Water | 0.00% | 0.05% | 0.10% | 1.00% | 2.00% | 0.05% | 0.10% | 1.00% | 2.00% |
| pH | 6.28 | 4.26 | 3.79 | 3.60 | 3.45 | 6.64 | 6.72 | 6.67 | 6.93 |
| Conductivity | 34.3 | 46.1 | 56.1 | 123.7 | 154.5 | 33.0 | 25.0 | 27.0 | 28.0 |
| (uS · cm⁻¹) | | | | | | | | | |
| Density (at 22 C.) | 0.9980 ± 0.0005 | 0.994 ± 0.001 | 0.994 | 0.995 | 0.994 | 0.994 ± 0.010 | 0.997 | 0.998 | 1.002 |
| (g · cm⁻¹) | | | | | | | | | |
| Viscosity (25 C.) | 0.91 | 0.92 | 0.91 | 1.01 | 1.07 | 0.91 | 0.91 | 0.94 | 1.04 |
| (cP) | | | | | | | | | |
| Turbidity (Abs, | (Reference) | 0.000 | 0.001 | 0.103 | 0.28 | 0.005 | 0.007 | 0.003 | 0.055 |
| =410 nm) | | | | | | | | | |
| Visual Inspection | Clear | Clear | Clear | Hazy | Turbid | Clear | Clear | Clear | Hazy |
| Surface Tension | 72 | 72 | 72 | 72 | 73 | 72 | 72 | 73 | 73 |
| (mN · m⁻¹) | | | | | | | | | |
| Osmolality | 0 | 0 | 0 | 15 | 28 | 0 | 0 | 6 | 15 |
| (mOsm/kg) | | | | | | | | | |

Tables 1-3 report the results of the physico-chemical test, wherein a notable effect is manifested in the density measurements, that presence of dissolved cyclodextrins at low concentration (0.05%) has a density-decreasing effect of purified water, and this phenomenon occurs also in the case of both L-Arginine and nicotinic acid complexes in low (0.05%) concentration. At higher concentration (0.5 and 1.0% solutions), however, this effect does not show up due to the higher solid content which evidently increase the density of the liquids.

The density measurements were repeated using tap water and it was found that the above-mentioned phenomenon does not occur probably due to the perturbating presence of ions in tap water. However, it may not be established exactly which ionic species (Mg2+, Ca2+, Na+) causes this perturbation. The results are shown in Table 4.

TABLE 4
Density of alpha-and gamma-CD solutions prepared with Tap water

| Tap water | | Alpha Cyclodexrin | | | |
|---|---|---|---|---|---|
| Concentration | No Additive | 0.05% | 0.10% | 1.00% | 2.00% |
| Density (at 22 C.) | 0.9987 ± 0.005 | 0.9992 ± 0.007 | 0.9994 ± 0.004 | 1.0021 ± 0.007 | 1.0088 ±0 .005 |
| (g · cm⁻¹) | | | | | |
| | | Gamma cyclodextrin | | | |
| Concentration | | 0.05% | 0.10% | 1.00% | 2.00% |
| Density (at 22 C.) | | 0.9997 ± 0.0007 | 1.0002 ± 0.0004 | 1.0035 ± 0.0007 | 1.0094 ± 0.0006 |
| (g · cm⁻¹) | | | | | |

### Example 3

### Effect of Cycodextrin Additives on Water Bonding Detected by IR Spectroscopy

Physical micro-structure studies of water, water-sugar interactions, and detection of sugar effects on increasing and decreasing water structure have preferentially employed infrared (IR) spectroscopy, and particularly near infrared (NIR) spectroscopy, as for example reported by Segtan et al. (see: Anal. Chem. 2001; 73, 3153-3161), and R. Giangiacomo (see: Food Chemistry, 2006, 96.3. 371-379.)

Hydration bond energies in pure waters and solutions of the same waters containing cyclodextrin compounds were assayed using IR spectroscopy in the near and middle infrared ranges. To record linear signals throughout an entire wavelength range, attenuation from water absorbance was minimized with a short optical length cuvette.

NIR range spectra were registered on a FOSS NIR Systems, Inc. 6500 spectrometer and Sample Transport Module (STM) using a 1 mm-sized cuvette. Transmission spectra were collected from 1100-2498 nm using a lead sulfide (PbS) detector and Vision 2.51 software (2001; FOSS NIRSystems, Inc.)

A Perkin-Elmer Spectrum 400 FT-NIR/FT-IR spectrometer and UATR (Universal Attenuated Total Reflectance; ZnSe-diamond crystal, 1× flat top plate) sample handling unit were used to obtain spectra across 2500-15385 nm (reported as 4000-650 cm⁻¹). Measurements were performed at 24 C using a triglycine-sulfate (TGS) detector and Spectrum ES 6.3.2 software (PerkinElmer, 2008).

Three samples of water were used in the present study. A first water sample was purified by reverse osmosis, carbon filtration, ultraviolet light exposure, membrane filtration to 0.2 micron absolute, and ozonation. Second and third water samples were not purified. Capillary electrophoresis revealed similar ionic components but at different concentrations between the three waters.

The following cyclodextrins were added to the above described water samples at a concentration range of 0.1%-5% w/w:

α-cyclodextrin (αCD also denoted as ACD), Lot. No. CYL-2322.
β-cyclodextrin (βCD also denoted as BCD). Lot. No. CYL-2518/2.
γ-cyclodextrin (γCD also denoted as GCD), Lot. No. CYL-2323.
2-hydroxypropyl-β-cyclodextrin (HPβCD, HPBCD), DS*=3.5, Lot. No. CYL-2232.
2-hydroxypropyl-γ-cyclodextrin (HPCD, HPGCD), DS*=4.8, Lot. No. CYL-2258.
carboxymethyl-β-cyclodextrin (CMBCD), Lot. No. CYL-2576.
quaternary-ammonium-β-cyclodextrin (QABCD).

For some examples, various inclusion complexes were formed between cyclodextrins and complex-forming bioactive agents, including the amino acids L-arginine and L-carnitine and the vitamin niacinamide (also known as nicotinamide). All reagents were of analytical purity. For some examples, L-arginine and nicotinamide were added in free form and alternatively in a cyclodextrin-complexed (molecularly entrapped) form to assess independent and co-dependent activities of a cyclodextrin and a bioactive agent. Concentrations of above additives in free form, and as cyclodextrin inclusion complex forms, were in the range of 0.1% to 5.0% w/w.

### Observations:

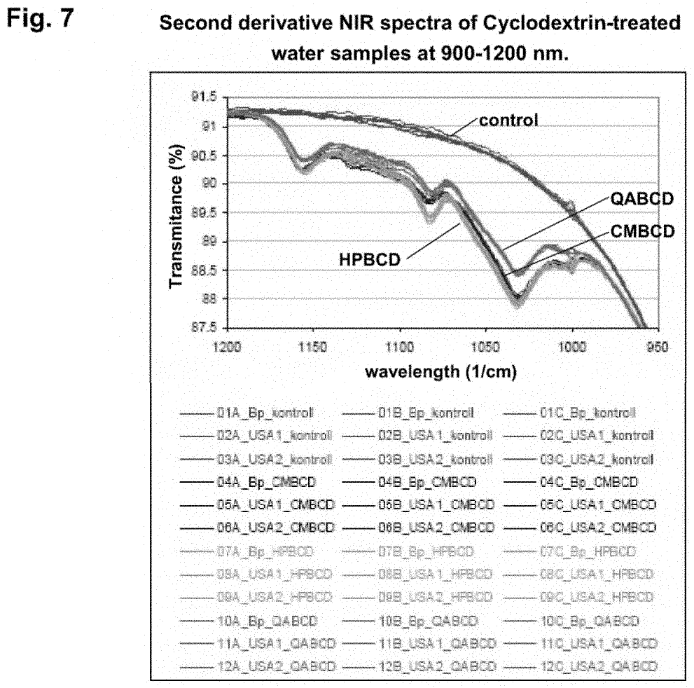

FIG. 7 shows second-derivative NIR spectra for the wavelength region 900-1200 nm. The results show water-bond interactions are significantly modified by addition of QABCD, and further significantly modified by addition of CMBCD and HPBCD.

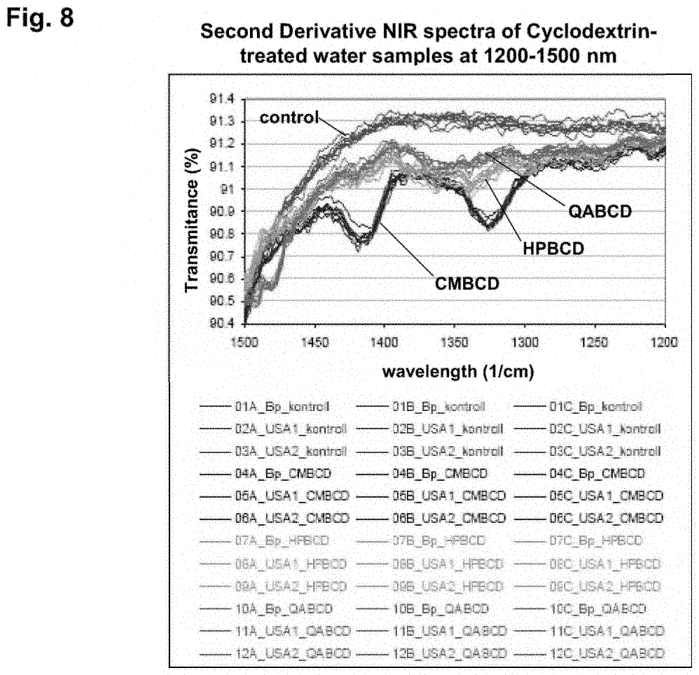

FIG. 8 shows second-derivative NIR spectra shown for 1200-1500 nm. The results show water-bond interactions are significantly modified by addition of QABCD and HPBCD, and further significantly modified by addition of CMBCD.

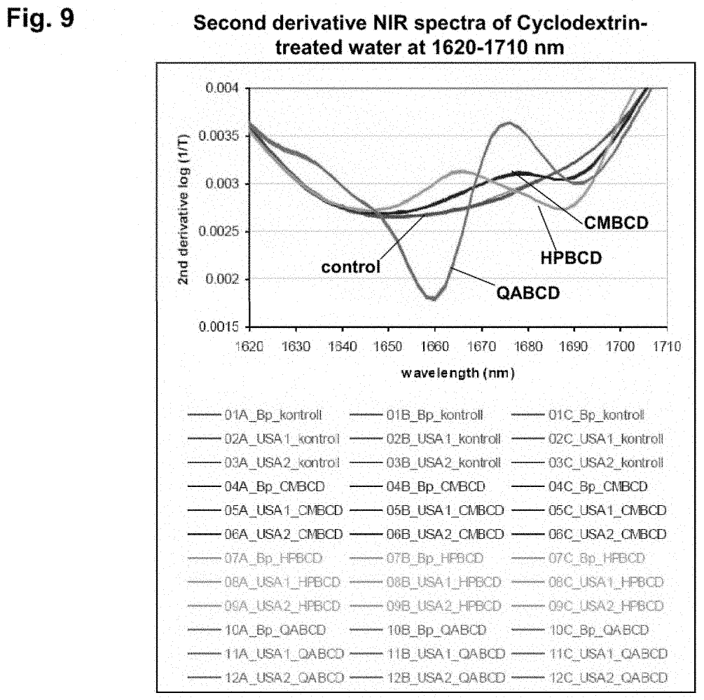

FIG. 9 shows second-derivative NIR spectra shown for 1620-1710 nm. The results show water-bond interactions are significantly modified by addition of CMBCD, QABCD, and HPBCD.

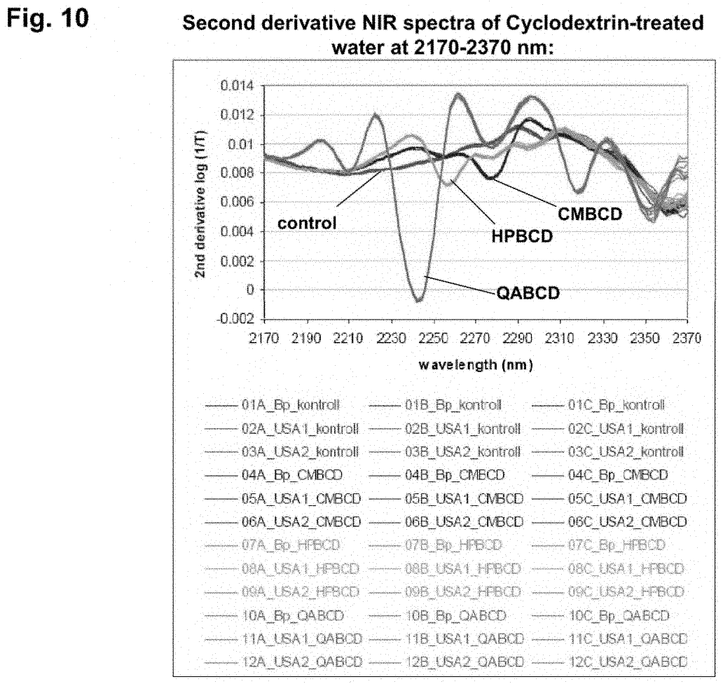

FIG. 10 shows second-derivative NIR spectra shown for 2170-2370 nm. The results show water-bond interactions are significantly modified by addition of CMBCD and HPBCD, and further significantly modified by addition of QABCD.

As shown in FIGS. 7-10, addition of cyclodextrins alters molecular bonding interactions of the aqueous medium. Referring particularly to FIG. 9, refined NIR spectra derivatives in the wavelength range of 1620-1770 nm show the carbon hydrogen bond related alterations involve CH3- CH2- and CH— groups of cyclodextrin additives. The significant spectral changes occurring in each cyclodextrin-treated water sample indicate the modified micro-structure of hydrogen bonds governed cluster systems in bulk water. This effect was largest in the water samples treated with charged quaternary-ammonium-β-cyclodextrins (QABCD), as shown for example in FIGS. 9 and 10.

### Example 4

### Acceleration of Plant Embryo Germination

Wheat seeds (Triticum aestivum) were germinated using USA I, USA II, and BP I waters described for Example 2. Germination rate using un-supplemented (control) water was compared to that with the same water variously supplemented with a cyclodextrin component, and/or a bioactive agent, as an active component of cellular hydration. For each condition, ten seeds were placed in continuous water contact in a Petri-type dish kept at 25 C in 12 hr light/dark cycles. Photometric images were recorded on days 1 to 6 after seeding. The percentage of seeds germinated was calculated and compared as a function of time and of the applied additive concentrations.

Water samples for seed germination were used alone with no additive, or containing cyclodextrins, or containing clathrate inclusion complexes of cyclodextrin with L-arginine or with nicotinamide (both obtained from Sigma Chemical Co.; St. Louis, Mo.), or with L-carnitine (from Lonza AG; Switzerland). Additives were included at 0.1 and 5. % (w/w). Additive solutions were prepared fresh on the day of germination start.

Parent cyclodextrins α-cyclodextrin (ACD), β-cyclodextrin (BCD), and γ-cyclodextrin (GCD), were obtained from Wacker Chemie (Munich, Germany). The following derivatized cyclodextrins were obtained from Cyclolab Ltd. (Budapest, Hungary): hydroxypropylated-beta-cyclodextrin (DS˜3) (HPBCD), carboxymethylated-β-cyclodextrin (DS˜3.5) (CMBCD), 2-hydroxy-3-N,N,N-trimethylamino)propyl-β-cyclodextrin chloride (DS˜3.6) (QABCD).

### Observations:

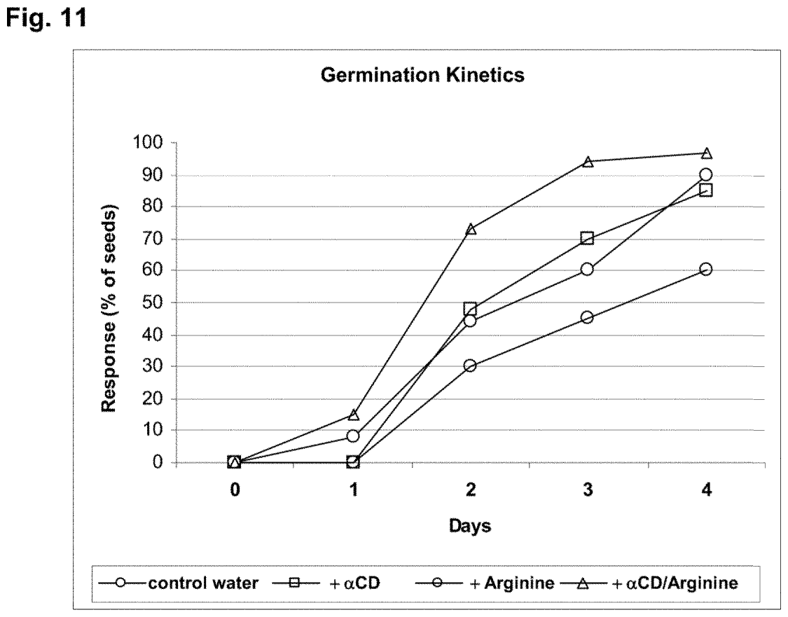

Germination kinetics in control and additive-modified water under identical conditions were quantified as the percentage of the seeds having a sprout. Each determination consisted of 100 seeds for each parameter. Results are reported in Table 5, below, and in FIGS. 11-13.

### A) Cyclodextrin/L-Arg Inclusion Complex Increases Seed Germination.

TABLE 5
Effect of α-cyclodextrin and L-arginine
on wheat seed germination rate
(values = percentage of total seeds)

| | control | α-Cyclodextrin, | L-Arg, | α-CD/L-Arg |
|---|---|---|---|---|
| Days | (water) | 0.5% | 0.5% | inc. complex |
| 0 | 0 | 0 | 0 | 0 |
| 1 | 8 | 0 | 0 | 15 |
| 2 | 44 | 48 | 30 | 73 |
| 3 | 60 | 70 | 45 | 94 |
| 4 | 90 | 85 | 60 | 97 |

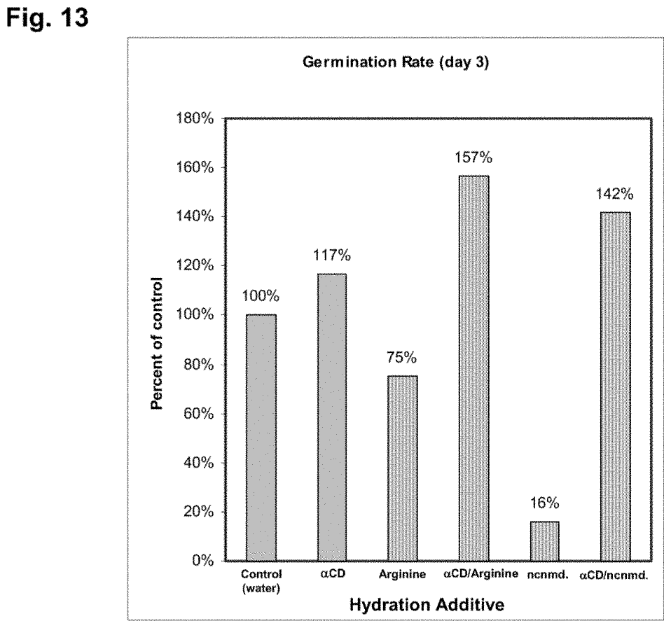

Table 5 shows comparative effects on the germination of wheat seeds of 0.5% w/w α-CD, 0.5% w/w L-arginine (L-Arg), and 0.5% w/w of an α-CD/L-arginine inclusion complex, each dissolved in USA I water. The above-tabulated results indicate that, compared to pure water lacking any additive (control), wheat seed germination rate is much higher in water including 0.5% (w/w) inclusion complex between α-cyclodextrin and L-arginine (αCD/L-Arg inc. complex). In addition, the results in Table 5 indicate that wheat seed germination rate is much higher in water including inclusion complex between α-cyclodextrin and L-arginine (αCD/L-Arg inc. complex) compared to water including 0.5% (w/w) α-cyclodextrin (αCD) as an additive alone, and also compared to water including 0.5% (w/w) L-arginine (L-Arg) as an additive alone. Thus, the results indicate a complex of α-cyclodextrin and L-arginine has a synergistic effect on increasing seed germination rate, which is not shown by either individual component of the complex used as a solitary additive. Results of Table 5 are also shown in FIGS. 11 and 13.

### B) Cyclodextrin/nicotinamide Inclusion Complex Increases Seed Germination.

TABLE 6
Effect of α-cyclodextrin and nicotinamide on
wheat seed germination rate

| | Control | α-Cyclodextrin, | nicotinamide, | α-CD/nicot. |
|---|---|---|---|---|
| Days | (water) | 0.5% | 0.5% | inc. complex |
| 0 | 2 | 0 | 0 | 0 |
| 1 | 9 | 11 | 0 | 0 |
| 2 | 50 | 18 | 4 | 65 |
| 3 | 62 | 68 | 10 | 88 |
| 4 | 90 | 92 | 60 | 100 |

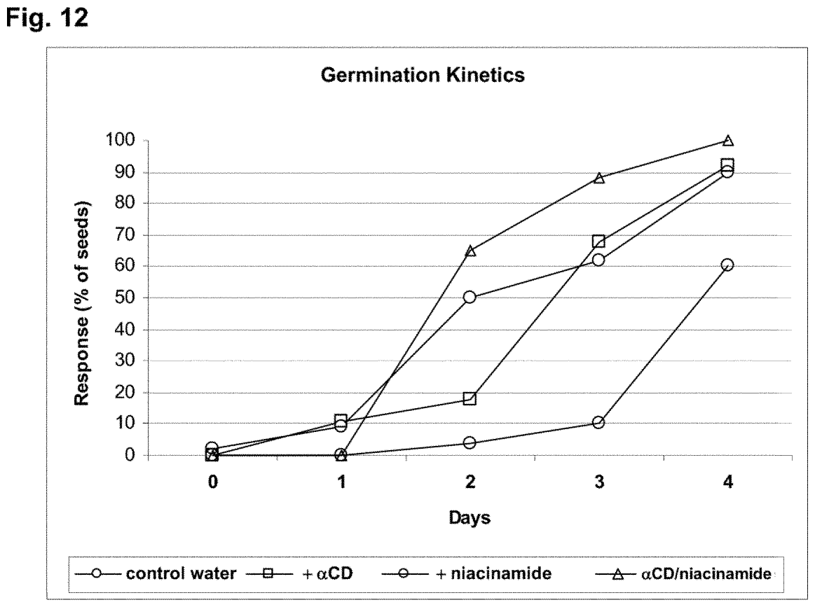

Table 6 shows comparative effects on the germination of wheat seeds of 0.5% w/w α-cyclodextrin, 0.5% w/w nicotinamide, and 0.5% w/w of an α-cyclodextrin/nicotinamide inclusion complex (αCD/nicot. inc. Complex), each dissolved in USA I water. The above-tabulated results indicate that, compared to pure water lacking any additive (control), wheat seed germination rate is much higher in water including inclusion complex between α-cyclodextrin and nicotinamide. In addition, the results in Table 6 indicate that wheat seed germination rate is much higher in water including inclusion complex between α-cyclodextrin and nicotinamide (αCD/nicot. inc. complex) compared to water including α-cyclodextrin (αCD) as an additive alone, and also compared to water including nicotinamide as an additive alone. Thus, the results indicate that when used as an inclusion complex, α-cyclodextrin and nicotinamide have a synergistic biological activity that significantly increases seed germination rate. Such biological activity was not demonstrated by either individual component of the complex used as a solitary additive. Results of Table 6 are also shown in FIGS. 12 and 13.

C) Qualitatively similar results as those reported in Tables 5 and 6, and FIGS. 11-13, were obtained using USA II and BP I water for germination. Thus, in particular, cyclodextrin inclusion complexes containing L-arginine, or alternatively containing nicotinamide, when dissolved in USA II or alternatively in BP I water, each significantly increased wheat seed germination rate, as shown above using USA I water.
D) Lengths of sprouts (rate of sprout growth during germination) did not differ between conditions within a statistically significant confidence interval (P<0.05). This result indicates that cyclodextrins, and particularly cyclodextrin inclusion complexes, may be used selectively as active components of cellular hydration to promote a rate of seed germination without necessarily also affecting a sprout growth rate.

### Example 5

### Lifespan Extension of C. elegans in Hydration Modified Water

C. elegans nematodes were grown in petri-type dishes containing normal nutrient liquid media prepared alternatively with USA I water (described in Example 2) lacking any further additive component (control) or the same water supplemented with a parent α-, β-, or γ-cyclodextrin, and/or a bioactive agent, as an active component of cellular hydration. Fifty ±3 worms were transferred to each dish. Each condition was repeated in triplicate. Experiments were repeated for USA II and BP I waters described in Example 2.
Water additives:
A. Addition of parent α-, β- and γ-cyclodextrins.
B. Addition of L-arginine and nicotinamide.
C. Addition of inclusion complexes of cyclodextrins with L-arginine and nicotinamide.

### Observations:

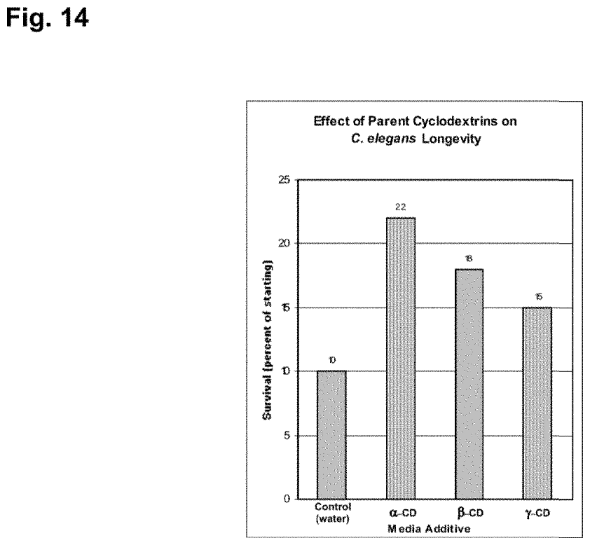

The results recorded are displayed below in Tables 7-9 and further presented in FIGS. 14-18.

TABLE 7
Effect of cyclodextrins on C. elegans longevity

| | Animals alive, % of initial | | | |
|---|---|---|---|---|
| | (N = 50) | | | |
| Life Span | Control | α-Cyclodextrin, | β-Cyclodextrin, | γ-Cyclodextrin, |
| (dfays) | (water) | 0.1% | 0.1% | 0.1% |
| 10 | 92 | 100 | 100 | 100 |
| 15 | 10 | 20 | 18 | 13 |
| 18 | 0 | 2 | 0 | 2 |

Table 7 reports the percentage of animals surviving to midlife (10 days), advanced age (15 days) and old age (18 days), in media variably containing a parent α-, β-, and γ-cyclodextrin as an active component of cellular hydration. In this example, parent cyclodextrins were added at a concentration of 0.1% w/w to nutritive media dissolved in USA I water.

Consistent with all previous studies, normal C. elegans animals in the present example survived two weeks in normal media. Each of the parent cyclodextrins markedly increased C. elegans survival (percentage alive) at advanced lifespan ages (days 10-15). Further, α-cyclodextrin and γ-cyclodextrins significantly increased the number of animals surviving to old ages, i.e., after day 15. The results are also represented graphically in FIG. 14, which compares the cumulative percentages of animals surviving to 15 and 18 days in media containing each additive parent cyclodextrin. The results show parent cyclodextrins, particularly α- and β-cyclodextrin, may be used as an active component of cellular hydration to improve biological function in a live animal. Biological mechanisms supporting advanced aging may include improvement of broad spectrum cellular activity during aging, or alternatively by selectively activating slow-aging cellular activity pathways. Clathrate-induced increases in water structure, hydration of cellular components, and diffusivity of bioactive cellular components, including inter- and intra-cellular signals, may all contribute to the overall effects of cyclodextrins on organism survival.

TABLE 8
Effect of chemically-modified cyclodextrins C. elegans longevity

| | Animals alive, % of initial | | | |
|---|---|---|---|---|
| | (N = 50) | | | |
| | | | | Quaternary |
| Life Span | Control | HP- | Carboxymethyl- | ammonium- |
| (days) | (water) | β-Cyclodextrin | β-Cyclodextrin | β-Cyclodextrin |
| 10 | 90 | 96 | 94 | 98 |
| 15 | 8 | 17 | 11 | 13 |
| 18 | 0 | 0 | 0 | 2 |

Table 8 reports the percentage of animals surviving to midlife (10 days), advanced age (15 days) and old age (18 days), in media variably containing a derivatized α-, β-, and γ-cyclodextrin as an active component of cellular hydration. In this example, derivatized cyclodextrins were added at 0.1% w/w to nutritive media dissolved in USA I water.

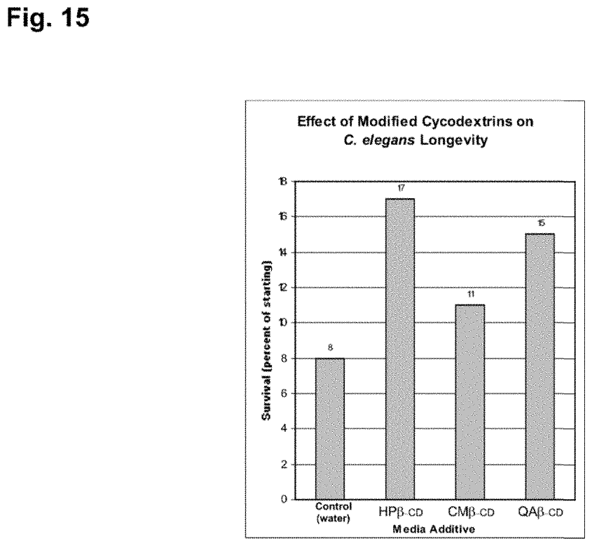

HP-, carboxymethyl-, and quaternary ammonium-derivatives of β-cyclodextrin had only slight effect on the initial survival of C. elegans to 10 days, as listed in Table 8. In contrast, significant increases in survival were observed at advanced ages (15 days), but not at old ages (18 days). The results are also shown graphically in FIG. 15, which compares the cumulative percentages of animals surviving to 15 and 18 days in media containing each additive derivatized cyclodextrin. The results indicate derivatized cyclodextrins may be used as an active component of cellular hydration to improve biological function in a live animal.

TABLE 9
Effect of cyclodextrin complexes on C. elegans longevity

| | Animals alive, % of initial | | | |
|---|---|---|---|---|
| | (N = 50) | | | |
| Life Span | Control | α-CD/ | α-CD/ | α-CD/ |
| (days) | (water) | L-Arg | L-carnitine | nicotinamide |
| 10 | 94 | 97 | 98 | 100 |
| 14 | 9 | 22 | 10 | 24 |
| 18 | 0 | 2 | 0 | 3 |

Table 9 reports the percentage of animals surviving to midlife (10 days), advanced age (14 days), and old age (18 days), in nutritive media dissolved in USA I water variably supplemented with a cyclodextrin inclusion complex at 0.1% w/w, as an active component of cellular hydration. In this example, inclusion complexes contained α-cyclodextrin and a bioactive agent, particularly L-arginine, L-carnitine, or niacinamide.

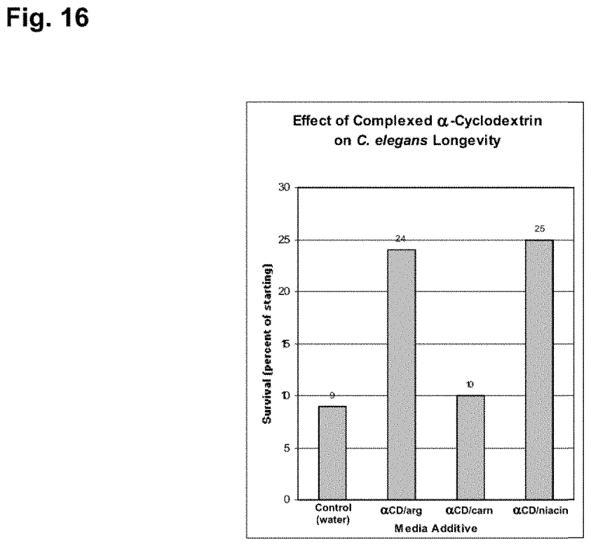

As in previous examples, C. elegans animals in un-supplemented media survived two weeks. α-Cyclodextrin complexes with L-arginine and niacinamide more than doubled C. elegans survival at advanced ages (day 14), and further permitted a small but significant number of animals to survive to an old age, to which no animal survived in nutritive media alone. In contrast, α-cyclodextrin complexes with L-carnitine had little or no significant effect on C. elegans survival. Similarly, L-arginine and nicotinamide added alone to the culture media without α-cyclodextrin had little effect on C. elegans survival. Results are also shown graphically in FIG. 16, which compares the cumulative percentages of animals surviving to 14 and 18 days in media containing each cyclodextrin inclusion complex as an additive. The results indicate α-cyclodextrin inclusion complexes, particularly complexes with L-arginine and niacinamide, may be used as an active component of cellular hydration to improve biological function in a live animal.

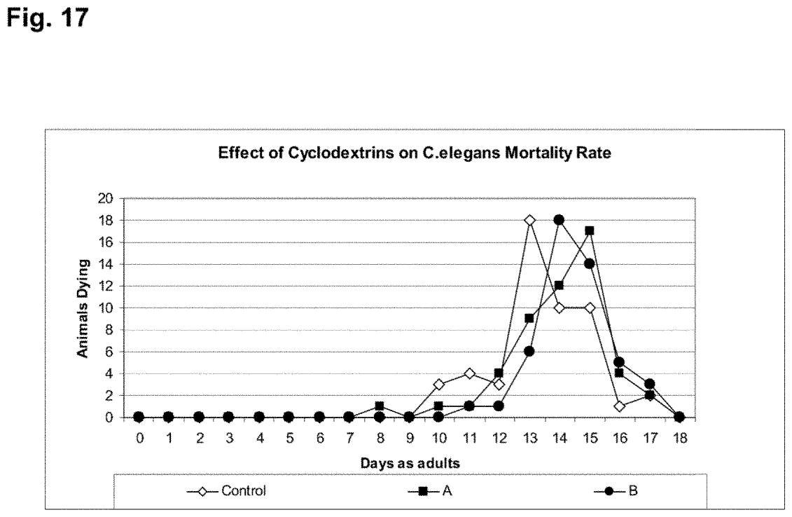

As further shown in FIG. 17, an inclusion complex of α-cyclodextrin and L-arginine (data series A; 1:1 complex, dissolved at 0.1% w/w in media made with USA I water), and an inclusion complex of α-cyclodextrin and niacinamide (data series B; 1:1 complex, dissolved at 0.1% w/w in media made with USA I water) can decrease the mortality rate of C. elegans worms. FIG. 17 shows the number of animals dying on each day for each media condition, wherein the control data series is media made with USA I water and lacking a further additive or supplement. The results show complexed forms of α-cyclodextrin may be used as an active component of cellular hydration to retard mortality of a live animal.

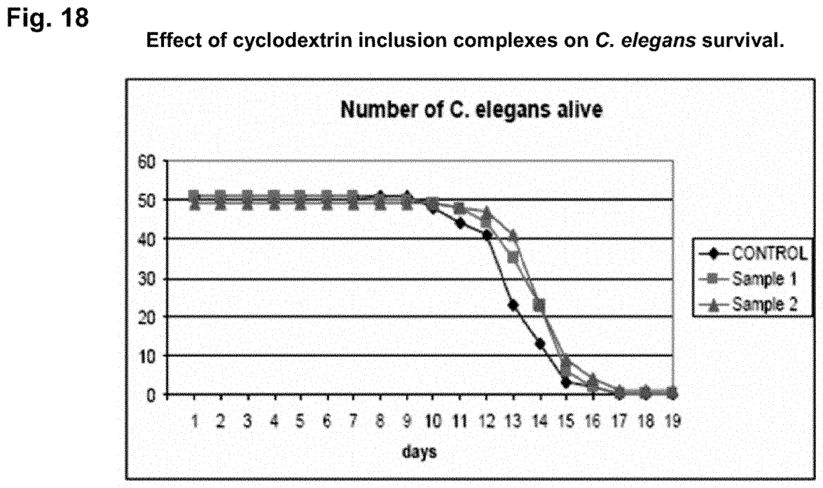

FIG. 18 alternatively represents the data of FIG. 17 as a survival curve for animals growing in normal media using USA I water (Control), or alternatively in media supplemented with a 1:1 inclusion complex of α-cyclodextrin and L-arginine (Sample 1); or in media supplemented with a 1:1 inclusion complex of cyclodextrin and niacinamide (Sample 2). Thus, the delay in mortality shown in FIG. 17 results in an older age of survival, the average age of survival (50% survival) increasing from nearly 13 days in normal media to nearly 14 days in media including a cyclodextrin inclusion complex as an active component of cellular hydration, which represents an 8% increase in lifespan.

### Example 6

### Lifespan Extension of C. elegans in Hydration Modified Water

C. elegans study performed for the present invention is a follow-up and repetition of the observations carried out earlier (referred as Test A here). The results of the current C. elegans study (Test B) were recorded with higher number of animals compared to the Test A (50 worms per treated groups versus 130 worms per groups). Nematodes were maintained and propagated on Nematode Growth Medium—(NGM) containing plates and fed with Escherichia coli OP50 bacteria. The C. elegans strain used in this study is Bristol (N2) as wild-type.

### Observations:

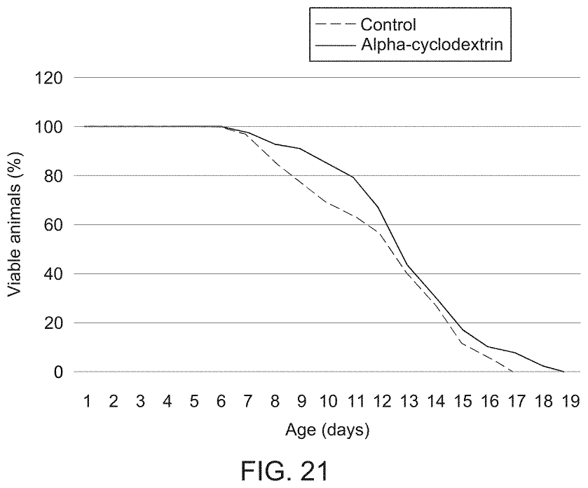

The results recorded are displayed below in Table 10 and further presented in FIGS. 21 & 22.

TABLE 10
Effect of alpha-cyclodextrin and its complexes on C. elegans lifespan

| C. elegans in Test A | | | | C. elegans in Test B | | | |
|---|---|---|---|---|---|---|---|
| Fraction of worms alive | | | | Fraction of worms alive | | | |
| | | 0.1% | | | | 0.1% | |
| | 0.1% | ACD- | 0.1% | | 0.1% | ACD- | 0.1% |
| | Alpha | nicotinic | ACD- | | Alpha | nicotinic | ACD- |
| Control | CD | acid | Arginine | Control | CD | acid | Arginine |
| 7-9% | 25% | 20-25% | 20-25% | 10% | 20% | 22-23% | 21-23% |

The present study (Test B) showed that the survival rate of control and ‘only water-treated’ animals on day 15 was 10%; hence, the two studies show results which are consistent with each other. Further, fair reproducibility of the 0.1% alpha-cyclodextrin-treated C. elegans lifespan was found. On the day 15 of experiments (which is equivalent in human 60 years of age) in 2009, about 20-25% of alpha-CD and alpha-CD complexes treated worms were found alive, while the same treatments resulted in about 20%-23% live fraction of C. elegans in the current study.

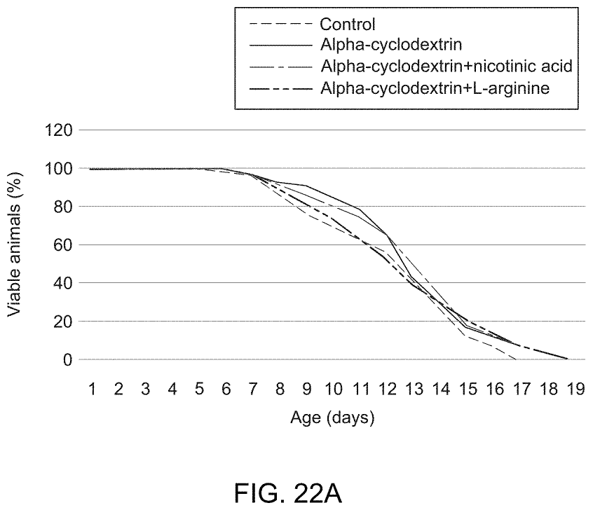

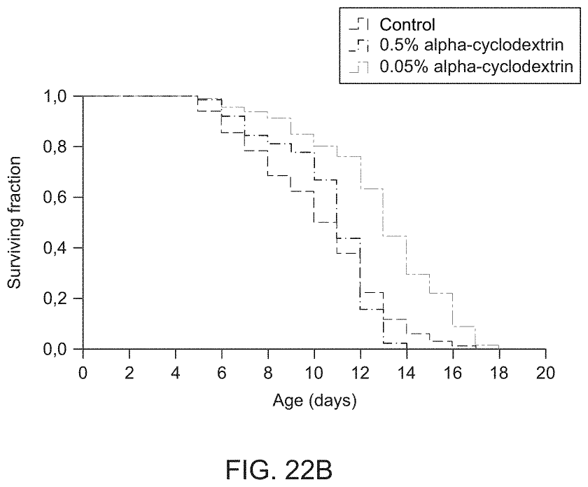

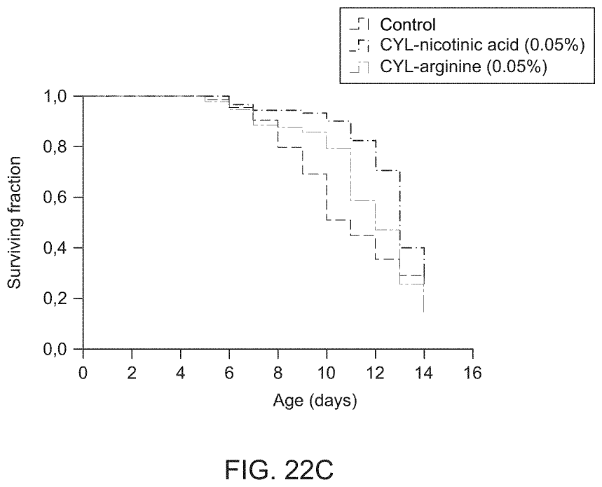

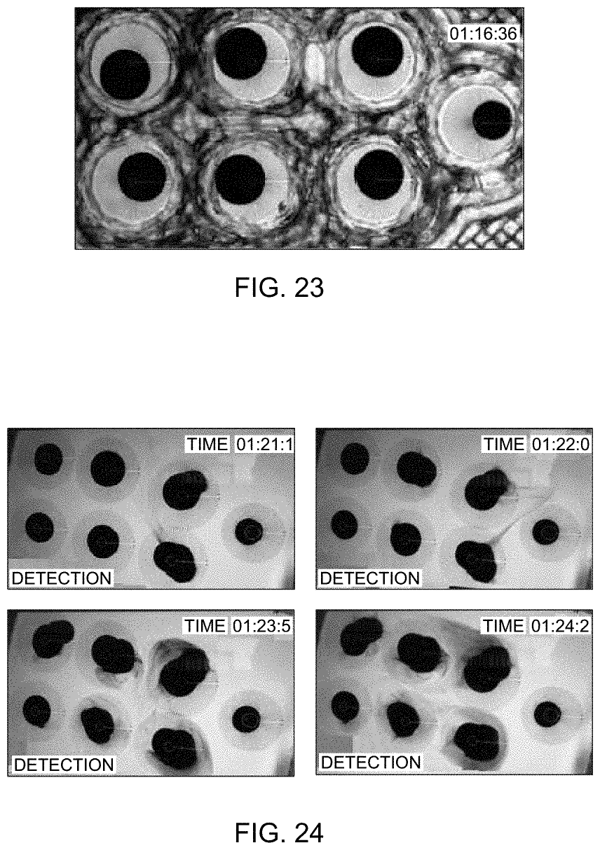

The C. elegans multi-cell testing (performed at the Institute of Genetics at University Eötvös Lorand, Vellai lab.) demonstrated a statistically significant enhancement in entire life span for the C. elegans treated with CD-enabled tap water compared to that of the control group. Moreover, it is noteworthy that the alpha-cyclodextrin treated C. elegans appeared more active and vibrant during early to mid-cycle (between 8-13 days) of their lives. Results are also shown graphically in FIGS. 21-22.

FIG. 21, illustrates the lifespan of control and alpha-cyclodextrin-treated worms. The control C. elegans lived on a medium made with tap water. The treated worms were maintained on culture media made with 0.1% alpha-cyclodextrin, 0.1% alpha-CD/nicotinic acid and 0.1% alpha-CD/arginine complex containing tap water. The lifespan curves are shown in FIGS. 21 and 22a.

Water samples containing alpha-CD and its complexes had a highly positive effect on C. elegans during the early- and mid-cycle of their lives (during 8-13 days of their lives). The average lifespan of control animals was 12.33 days while for the alpha-CD treated ones was 13.25 days. Approximately 1 day survival of the nematodes is equivalent to 4 or 5 years in a human life.

The tests were repeated with lower and higher alpha-CD concentrations (0.05 and 0.5%) as well as complex solutions of 0.05% concentration. The lifespan curves are shown in FIGS. 22b and 22c, respectively. It is notable that the dose dependence of the lifespan elongation effect of cyclodextrin and its complexes is non-linear: the efficacy of 0.05% concentration surpasses that of the tested higher concentration samples. Nevertheless, the effect of alpha cyclodextrin was significant in all the three studied concentrations.

The Caenorhabditis elegans life span testing demonstrated a statistically significant enhancement in entire life span when treated with CD-enabled water, compared to the control group. It was observed that the alpha-cyclodextrin treated C. elegans appeared more active and vibrant during early to mid-cycle (between 8-13 days) of their lives. The effect was similarly beneficial when alpha cyclodextrin complexes (prepared of L-arginine or nicotinic acid) were applied.

### Example 7

### Effect of Cyclodextrins on Cell Hydration Using Xenopus Frog Oocytes

For Xenopus oocyte test, oocyte was harvested by anaesthetizing Xenopus laevis with 0.15% MS-222 in water for 15 min. They were then kept on ice for another 15 min before ovarectomy was performed. Ovaries were incubated in collagenase (Worthington Type II, 10 mg/ml) in calcium free Barth's solution (CFBS, NaCl 88 mM, KCl 1 mM, MgSO₄ 0.8 mM, TRIS-HCl 5 mM, NaHCO₃ 2.4 mM). Following defolliculation, oocytes were rinsed in normal Barth's solution (MBS, NaCl 88 mM, KCl 1 mM, CaCl₂ 0.4 mM, Ca(NO₃)₂ 0.33 mM, MgSO₄ 0.8 mM, TRIS-HCl 5 mM, NaHCO₃ 2.4 mM) before they were transferred to 96-well plates. For nuclear injection of the different DNA's into the oocytes and for cytoplasmic injection of the mRNA encoding human Aquaporin-1 channels into the oocytes, the Roboocyte automated injection and recording system was used. (Human Aquaporine 1 cDNA cloned in expressing vector pGEM-T were purchased from Sino Biological Inc. Transcription to AQP1 mRNA was performed by an Ecocyte cooperation partner lab.) The mRNA injection volume was in the range 20-50 nl at a mRNA concentration of 100 ng/μl. After two to three days of incubation in Barth's solution supplemented with Gentamycin, water uptake of the Xenopus Oocytes through AQP1 channels was tested in a swelling assay using video microscopy.

All test compound mixtures (Alpha cyclodextrin (ACD) 0.05%, 0.1%, 0.5%; ACD-nicotinic acid complex, 0.05%, 0.1%, 0.5%, ACD-arginine complex, 0.05%, 0.1%, 0.5%) were prepared in purified water and were supported by Cyclolab in 500 ml amounts as well as a 500 ml purified water sample. Normal frog ringer (NFR, NaCl 90 mM, KCl 2 mM, CaCl₂ 2 mM, MgCl₂ 1 mM, HEPES 5 mM, osmolarity 200 mOsm/l) was used as control solution and was prepared freshly on the day of the experiments. All solutions were handled in double blind experiments. Compound mixtures as well as water controls were labelled as C1-C10. Then the swelling assay/video microscopy and data analysis was performed.

Water is a major component of the cell, it represents 70-95% of its weight. Water can cross lipid bilayers of all biological membranes by simple diffusion and the discovery of water channels, by Nobel-laurate Peter Agre in cells called aquaporins, provides a molecular explanation for the rapid and regulated transport of water across the lipid bilayers of cell membranes.

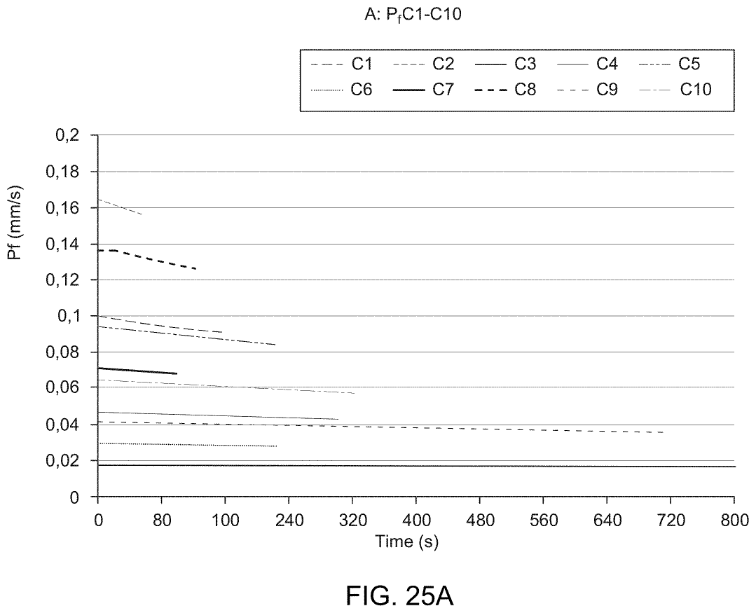

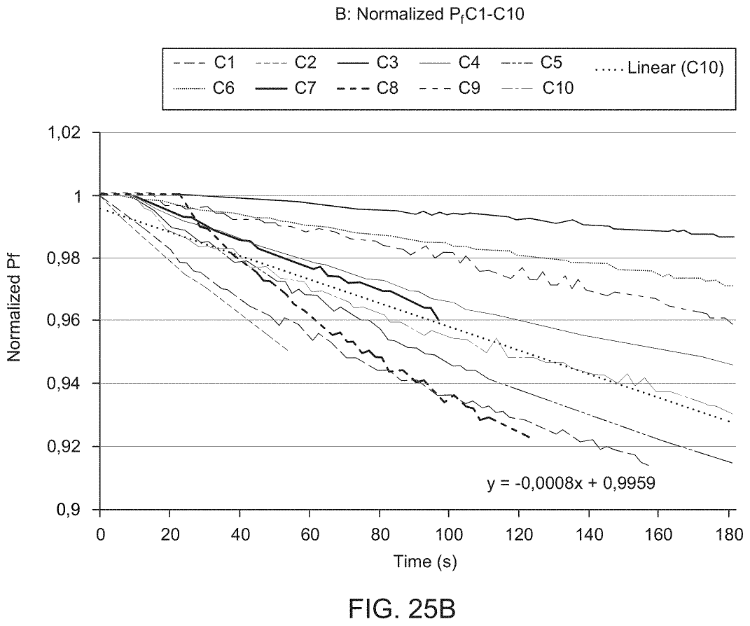

The study used the same biological system that was used by Peter Agre. Frog oocytes (eggs) are resistant to water permeation, as mother frogs lay their eggs in water. Peter Agre used genetic material by injecting ribonucleic acid into these oocytes, causing expression membrane integrated water channel proteins. So, the oocytes became permeable for water. Oocyte water channel testing method was used for the description of the effect of cyclodextrins on cellular water uptake through human aquaporin 1 (AQP1). The results of oocyte osmotic water permeability are illustrated on FIG. 24-25.

The results of the Xenopus oocyte test show that the highest water permeation was recorded to water solutions containing 0.05% alpha-cyclodextrin and 0.05% alpha-cyclodextrin/arginine complex. Surprisingly, the tested solutions with higher (0.1-0.5%) cyclodextrin content showed reduced water permeation compared to control tap water. Further, the single-celled oocyte test also indicates the positive effect of the same low concentration (0.05%) of cyclodextrins on the cellular water uptake.

### Example 8

### Effect of Cyclodextrins and Complexing Agent on Water Absorption Using Orbeez Beads

A set of observational experiments using different concentrations of the Cyclodextrin formula, complexing agents (arginine and niacin) and a control were performed. These experiments are visual in nature. Orbeez beads, made of super absorbent polymers and colored contact lenses which also absorb water, were selected for the experiment.

Equal amounts of Orbeez and Contact lenses were taken and placed/weighed using a calibrated scientific scale. 500 ml solutions of Cyclodextrin+Arginine and Niacin (our complexing agents) in 1% and 2% concentrations were mixed up. The different solutions with the test products were combined in petri dishes and were observed/photographed at standard time intervals: 30 mins., 1 hour, 3 hours. Tap water was used as the Control solution for Orbeez beads and the Control for the Contact lens solution was saline. 1% and 2% formulations of CD+Arg+Niacin were solubilized in saline for the contact lens test. The test products were removed from water and weighed after the 3-hour interval. The test products were photographed in a side by side comparison after the 3-hour interval.

### Observations:

TABLE 11
Orbeez beads Experiment

| Orbeez beads | |
|---|---|
| (experiment | |
| time) | Observation |
| Prior to | Orbeez beads are hard yet absorbent and multi-colored. |
| Experiment | |
| At 1 Hr | Measurement clearly shows 1% and 2% solution more |
| | defined and larger than the control group |
| At 3 Hrs | At 3 Hours, the Orbeez beads in the 2% solution |
| | exhibited the greatest growth compared to the control. |
| | The 2% solution of cyclodextrin-Arginine and Niacin |
| | produced a weight 40% greater than the control. Orbeez |
| | beads at 3 hours exhibited significantly greater absorption |
| | in the 2% cyclodextrin solution. |

The experiment is an illustration of the enhanced water absorbing effect that cyclodextrin complexes offer in comparison to standard water for the Orbeez test and saline for the Contact lens test. Furthermore, we believe that these results can be extrapolated to consumed beverages and their ability to penetrate biological cells, thus improving a human body's hydration and its ability to absorb liquids and nutrients.

The invention may also be described by the following numbered paragraphs:

1. A beverage composition that promotes cellular hydration when ingested by a multicellular organism, comprising:

a carbohydrate clathrate component that includes cyclodextrin, in a concentration of 0.01-5% w/w;

a complex-forming compound, in a concentration that is less than the clathrate component;

an aqueous liquid component, chosen from the group consisting of still and carbonated aqueous liquids;

wherein an inclusion complex is formed with at least some of the clathrate component and at least some of the complex-forming compound; and

wherein the composition promotes cellular hydration of the multicellular organism when the multicellular organism ingests it.

2. The beverage composition of paragraph 1, wherein the ratio of clathrate component to complex-forming compound is in a range from about 5:1 to about 15:1.

3. The beverage composition of paragraph 1, wherein the complex-forming compound is selected from the group consisting of amino acids, including L-arginine, citrulline, creatine, taurine, nicotinic acid, nicotinamide, resveratrol, curcumin, thiamine, curcumin, polyphenols, dihydrocurcumin, spermidin, L-lysin, coenzymeQ10, delta-tocopherol, delphindin, caffeine, and guarna.

4. The beverage composition of paragraph 1, wherein the complex-forming compound is selected from the group consisting of any electrolyte, and specifically, from the group consisting of magnesium, sodium, potassium, chloride, calcium, phosphate, and bicarbonate.

5. The beverage composition of paragraph 1, wherein the composition causes cellular hydration in a multicellular organism when a multicellular organism ingests it.

6. The beverage composition of paragraph 1, wherein the multicellular organism is capable of intracellular water permeation, and the ingestion of the composition by the multicellular organism enhances the intracellular permeation.

7. The beverage composition of paragraph 6, wherein the multicellular organism contains aquaporins, and the cellular hydration is caused by interaction of the composition with the aquaporins.

8. The beverage composition of paragraph 7, wherein the cellular hydration is corroborated by a test that uses human-aquaporin-expressed frog oocytes.

9. The beverage composition of paragraph 8, wherein the test uses single cell Xenopus laevis human-aquaporin-expressed frog oocytes having expressed human aquaporin AGP1 water channels.

10. The beverage composition of paragraph 1, wherein the composition also promotes increased lifespan of the multicellular organism.

11. The beverage composition of paragraph 10, wherein the promotion of increased lifespan is corroborated by lifespan studies on C. elegans nematodes.

12. The beverage composition of paragraph 11, wherein the composition causes increased lifespan of the multicellular organism.

13. The beverage composition of paragraph 10, wherein the cause of increased lifespan is corroborated by lifespan studies on C. elegans nematodes.

14. The beverage composition of paragraph 1, wherein the cyclodextrin is chosen from the group consisting of alpha-, beta-, and gamma-cyclodextrins.

15. A beverage composition that promotes increased lifespan when ingested by a multicellular organism, comprising:

a carbohydrate clathrate component that includes cyclodextrin, in a concentration of 0.01-5% w/w;

a complex-forming compound, in a concentration that is less than the clathrate component;

an aqueous liquid component, chosen from the group consisting of still and carbonated aqueous liquids;

wherein an inclusion complex is formed with at least some of the clathrate component and at least some of the complex-forming compound; and

wherein the composition promotes increased lifespan of the multicellular organism when the multicellular organism ingests it.

16. The beverage composition of paragraph 15, wherein the promotion of increased lifespan is corroborated by lifespan studies on C. elegans nematodes.

17. The beverage composition of paragraph 16, wherein the composition causes increased lifespan of the multicellular organism.

18. The beverage composition of paragraph 17, wherein the cause of increased lifespan is corroborated by lifespan studies on C. elegans nematodes.

19. A system that promotes cellular hydration when ingested by a multicellular organism, comprising:

a carbohydrate clathrate component that includes cyclodextrin, in a concentration of 0.01-5% w/w;

a complex-forming compound, in a concentration that is less than the clathrate component;

an aqueous liquid component, chosen from the group consisting of still and carbonated aqueous liquids;

wherein an inclusion complex is formed with at least some of the clathrate component and at least some of the complex-forming compound; and

wherein the composition promotes cellular hydration when a multicellular organism ingests it.

20. The system of paragraph 19, wherein the ratio of clathrate component to complex-forming compound is in a range from about 5:1 to about 15:1.

21. The system of paragraph 20, wherein the complex-forming compound is selected from the group consisting of amino acids, including L-arginine, citrulline, creatine, taurine, nicotinic acid, nicotinamide, resveratrol, curcumin, thiamine, curcumin, polyphenols, dihydrocurcumin, spermidin, L-lysin, reservatrol, coenzymeQ10, delta-tocopherol, delphindin, caffeine, and guarna.

22. The system of paragraph 20, wherein the complex-forming compound is selected from the group consisting of any electrolyte, and specifically, from the group consisting of magnesium, sodium, potassium, chloride, calcium, phosphate, and bicarbonate.

23. A method of promoting increased cellular hydration in a multicellular organism that is capable of intracellular water permeation, comprising:

causing the multicellular organism to ingest an aqueous solution that contains an amount of a carbohydrate clathrate component; and

enhancing the intracellular permeation.

24. The method of paragraph 23, wherein the multicellular organism contains aquaporins, and the causing step involves interaction of the composition with the aquaporins.

25. The method of paragraph 24, wherein the cellular hydration is corroborated by a test that uses human-aquaporin-expressed frog oocytes.

26. The method of paragraph 25, wherein the test uses single cell Xenopus laevis human-aquaporin-expressed frog oocytes having expressed human aquaporin AGP1 water channels.

27. The method of paragraph 23, wherein the multicellular organism has lipid bilayer constituents, and further including forming non-covalent inclusion complexes between the clathrate component and the lipid bilayer constituents.

28. The method of paragraph 23, wherein the multicellular organism also has phospholipids chosen from the group consisting of glycosphingolipids, sphingomyelin, phosphatidylcholine, phosphatidyl ethanolamine.

29. The method of paragraph 23, wherein the phospholipids are linear.

30. The method of paragraph 23, wherein the multicellular organism also includes membrane lipids and proteins, and the causing results in temporary disintegration of membrane lipids and proteins.

31. The method of paragraph 23, wherein the multicellular organism includes lipid packing, and the causing results in loosening of lipid packing.

32. The method of paragraph 23, wherein the multicellular organism includes membrane proteins, and the causing results in untightening of membrane proteins in an area that includes the membrane proteins.

33. The method of paragraph 23, wherein the multicellular organism includes protein structure and protein function, and the causing results in changes in the protein structure and protein function.

34. The method of paragraph 23, wherein the multicellular organism includes membrane lipids, lipid packing, membrane proteins, protein structure and protein function, and the causing results in temporary disintegration of the membrane lipids, loosening of the lipid packing, untightening of the membrane proteins, and changes in the protein structure and the protein function.

35. The method of paragraph 27, wherein multicellular organism includes cellular layers, and the temporary disintegration of membrane lipids and proteins leads to enhanced membrane permeation of nutrients and water into the cellular layers.

36. The method of paragraph 23, wherein the multicellular organism includes cholesterols, the clathrate component includes beta cyclodextrin, and the causing results in binding to the cholesterols.

37. A method of promoting increased cellular hydration in a multicellular organism that includes water, comprising:

causing the multicellular organism to ingest an aqueous solution that contains an amount of a carbohydrate clathrate component; and

decreasing the density of at least some of the water in the aqueous solution.

38. The method of paragraph claim 37, wherein the multicellular organism also contains aquaporins, and the causing step involves interaction of the composition with the aquaporins.

39. The method of paragraph 38, wherein the cellular hydration is corroborated by a test that uses human-aquaporin-expressed frog oocytes.

40. The method of paragraph 39, wherein the test uses single cell Xenopus laevis human-aquaporin-expressed frog oocytes having expressed human aquaporin AGP1 water channels.

41. The method of paragraph 37, wherein the multicellular organism has lipid bilayer constituents, and further including forming non-covalent inclusion complexes between the clathrate component and the lipid bilayer constituents.

42. The method of paragraph 37, wherein the multicellular organism also has phospholipids chosen from the group consisting of glycosphingolipids, sphingomyelin, phosphatidylcholine, phosphatidyl ethanolamine.

43. The method of paragraph 37, wherein the phospholipids are linear.

44. The method of paragraph 37, wherein the multicellular organism also includes membrane lipids and proteins, and the causing results in temporary disintegration of membrane lipids and proteins.

45. The method of paragraph 37, wherein the multicellular organism includes lipid packing, and the causing results in loosening of lipid packing.

46. The method of paragraph 37, wherein the multicellular organism includes membrane proteins, and the causing results in untightening of membrane proteins.

47. The method of paragraph 37, wherein the multicellular organism includes protein structure and protein function, and the causing results in changes in the protein structure and protein function.

48. The method of paragraph 37, wherein the multicellular organism includes membrane lipids, lipid packing, membrane proteins, protein structure and protein function, and the causing results in temporary disintegration of the membrane lipids, loosening of the lipid packing, untightening of the membrane proteins, and changes in the protein structure and the protein function.

49. The method of paragraph 44, wherein multicellular organism includes cellular layers, and the temporary disintegration of membrane lipids and proteins leads to enhanced membrane permeation of nutrients and water into the cellular layers.

50. The method of paragraph 37, wherein the multicellular organism includes cholesterols, the clathrate component includes beta cyclodextrin, and the causing results in binding to the cholesterols.

51. A method for increasing hydration of cell system to promote cellular hydration in a multicellular organism when the mixture is ingested, the multicellular organism containing membrane lipids, lipid packing and membrane proteins, protein structure and protein function, and membrane permeation of nutrients and water, the method comprising the step of:

causing the multicellular organism to ingest an aqueous solution that contains an amount of a carbohydrate clathrate component; and

changing the multicellular organism by (i) temporary disintegration of the membrane lipids, (ii) loosening of the lipid packing and membrane proteins, and (iii) altering the protein structure and protein function, collectively to enhance membrane permeation of nutrients and water.

52. The method of paragraph 51, wherein the clathrate is alpha cyclodextrin, and further including the step of binding the alpha cyclodextrin to linear phospholipids in the human body, with the phospholipids chosen from the group consisting of glycosphingolipids, sphingomyelin, phosphatidylcholine, phosphatidyl ethanolamine

53. The method of paragraph 51, wherein the clathrate is beta cyclodextrin, and further including the step of binding to cholesterols.

Although the present invention has been shown and described with reference to the foregoing operational principles and preferred embodiments, it will be apparent to those skilled in the art that various changes in form and detail may be made without departing from the spirit and scope of the invention. The present invention is intended to embrace all such alternatives, modifications and variances that fall within the scope of the appended claims.

What is claimed is:

1. A method of promoting increased cellular hydration in a multicellular organism that has cells with cell membranes, lipid packing associated with the cell membranes, aquaporins, and is capable of intracellular water permeation, comprising:
causing the multicellular organism to ingest an aqueous solution that contains an amount of a carbohydrate clathrate component and, thereby, causing interaction of the aqueous solution with the aquaporins, and causing the clathrate component to physically change the lipid packing of the cell membranes; and
enhancing the intracellular permeation because of the causing step.

2. The method of claim 1, wherein, as a result of the causing and enhancing steps, increased cellular hydration can be corroborated by a test that uses human-aquaporin-expressed frog oocytes, and measures water uptake of the frog oocytes in a swelling assay.

3. The method of claim 2, wherein the test uses single cell Xenopus laevis human-aquaporin-expressed frog oocytes having expressed human aquaporin AGP1 water channels.

4. The method of claim 1, wherein the multicellular organism has lipid bilayer constituents, and further including forming non-covalent inclusion complexes between the clathrate component and the lipid bilayer constituents.

5. The method of claim 1, wherein the multicellular organism also has phospholipids chosen from the group consisting of glycosphingolipids, sphingomyelin, phosphatidylcholine, phosphatidyl ethanolamine.

6. The method of claim 5, wherein the phospholipids are linear.

7. The method of claim 1, wherein the multicellular organism also includes membrane lipids and proteins, and the causing results in temporary disintegration of membrane lipids and proteins.

8. The method of claim 1, wherein the multicellular organism includes lipid packing, and the causing results in loosening of lipid packing.

9. The method of claim 1, wherein the multicellular organism includes membrane proteins, and the causing results in untightening of membrane proteins in an area that includes the membrane proteins.

10. The method of claim 1, wherein the multicellular organism includes protein structure and protein function, and the causing results in changes in the protein structure and protein function.

11. The method of claim 1, wherein the multicellular organism includes membrane lipids, lipid packing, membrane proteins, protein structure and protein function, and the causing results in temporary disintegration of the membrane lipids, loosening of the lipid packing, untightening of the membrane proteins, and changes in the protein structure and the protein function.

12. The method of claim 4, wherein multicellular organism includes cellular layers, and the temporary disintegration of membrane lipids and proteins leads to enhanced membrane permeation of nutrients and water into the cellular layers.

13. The method of claim 1, wherein the multicellular organism includes cholesterols, the clathrate component includes beta cyclodextrin, and the causing results in binding to the cholesterols.

14. The method of claim 2, wherein the swelling assay uses video microscopy.
